(19) United States
(12) Patent Application Publication
(10) Pub. No.: US 2005/0137644 A1
(43) Pub. Date: Jun. 23, 2005

(54) Method and system for vagal blocking and/or vagal stimulation to provide therapy for obesity and other gastrointestinal disorders

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/032,652

(22) Filed: Jan. 8, 2005

Related U.S. Application Data
(63) Continuation-in-part of application No. 10/079,215, filed on Feb. 20, 2002, now Pat. No. 6,879,859.
Application No. 10/079,215 is a continuation of application No. 09/751,966, filed on Dec. 29, 2000, now Pat. No. 6,366,814.
Application No. 09/751,966 is a continuation of application No. 09/178,060, filed on Oct. 26, 1998, now Pat. No. 6,205,359.

(51) Int. Cl. A61N 1/18

(52) U.S. Cl. 607/40

(57) ABSTRACT

Method and system to provide therapy for obesity and gastrointestinal disorders such as FGIDs, gastroparesis, gastro-esophageal reflex disease (GERD), pancreatitis and ileus comprises vagal blocking and/or vagal stimulation. Vagal blocking may be in the afferent or efferent direction, and may be with or without stimulation pulses. Blocking may be provided by one of a number of different electrical blocking techniques. Electrical signals may be provided with an external stimulator in conjunction with an implanted stimulus-receiver, or an implanted stimulus-receiver comprising a high value capacitor for temporary power source. In one embodiment, the external stimulator may comprise an optional telemetry unit. The addition of the telemetry unit to the external stimulator provides the ability to remotely interrogate and change stimulation programs over a wide area network, as well as other networking capabilities,

This is a continuation of application Ser. No. 10/079,215, entitled “An external pulse generator for adjunct (add-on) treatment of obesity, eating disorders, neurological, neuropsychiatric, and urological disorders”, which is a continuation of application Ser. No. 09/751,966 filed Dec. 29, 2000, now U.S. Pat. No. 6,366,814 which is a Continuation-in-Part application of Ser. No. 09/178,060 filed Oct. 26, 1998, now U.S. Pat. No. 6,205,359. The prior applications and patents being incorporated herein by reference.

This Application is also related to commonly assigned U.S. Pat. No. 6,611,715, entitled “Apparatus and Method for Neuromodulation Therapy for Obesity and Compulsive Eating Disorders Using an Implantable Stimulus-receiver and an External Stimulator”. This patent is incorporated herein by reference.

### FIELD OF INVENTION

This invention relates generally to electrical stimulation therapy for medical disorders, more specifically to neuromodulation therapy comprising vagal blocking with or without vagal stimulation for providing therapy for obesity and other gastrointestinal (GI) disorders, with an external pulse generator (stimulator) adapted to be used with an implanted stimulus-receiver.

### BACKGROUND OF OBESITY AND RELATION TO VAGUS NERVE

Obesity is a significant health problem in the United States and many other developed countries. Obesity results from excessive accumulation of fat in the body. It is caused by ingestion of greater amounts of food than can be used by the body for energy. The excess food, whether fats, carbohydrates, or proteins, is then stored almost entirely as fat in the adipose tissue, to be used later for energy. Obesity is not simply the result of gluttony and a lack of willpower. Rather, each individual inherits a set of genes that control appetite and metabolism, and a genetic tendency to gain weight that may be exacerbated by environmental conditions such as food availability, level of physical activity and individual psychology and culture. Other causes of obesity also include psychogenic, neurogenic, and other metabolic related factors.

Obesity is defined in terms of body mass index (BMI), which provides an index of the relationship between weight and height. The BMI is calculated as weight (in Kilograms) divided by height (in square meters), or as weight (in pounds) times 703 divided by height (in square inches). The primary classification of overweight and obesity relates to the BMI and the risk of mortality. The prevalence of obesity in adults in the United States without coexisting morbidity increased from 12% in 1991 to 17.9% in 1998, and is still increasing.

Treatment of obesity depends on decreasing energy input below energy expenditure. Treatment has included among other things various drugs, starvation, and even stapling or surgical resection of a portion of the stomach. Surgery for obesity has included gastroplasty and gastric bypass procedure. Gastroplasty which is also known as stomach stapling, involves constructing a 15- to 30 mL pouch along the lesser curvature of the stomach. A modification of this procedure involves the use of an adjustable band that wraps around the proximal stomach to create a small pouch. Both gastroplasty and gastric bypass procedures have a number of complications.

The vagus nerve (which is the 10^{th} cranial nerve) plays a role in mediating afferent information from the stomach to the satiety center in the brain. The vagus nerve arises directly from the brain, but unlike the other cranial nerves extends well beyond the head. At its farthest extension it reaches the lower parts of the intestines. This is shown schematically in FIG. 1, and in more detail in FIG. 2.

In 1988 it was reported in the American Journal of Physiology, that the afferent vagal fibers from the stomach wall increased their firing rate when the stomach was filled. One way to look at this regulatory process is to imagine that the drive to eat, which may vary rather slowly with the rise and fall of hormone Leptin, is inhibited by satiety signals that occur when we eat and begin the digestive process (i.e., the prandial period). As shown schematically in FIG. 3, these satiety signals both terminate the meal and inhibit feeding for some time afterward. During this postabsorptive (fasting) period, the satiety signals slowly dissipate until the drive to eat again takes over

The regulation of feeding behavior involves the concentrated action of several satiety signals such as gastric distention, the release of the gastrointestinal peptide cholecystokinin (CCK), and the release of the pancreatic hormone insulin. The stomach wall is richly innervated by mechanosensory axons, and most of these ascend to the brain via the vagus nerve(s) 54. The vagus sensory axons activate neurons in the Nucleus of the Solitary Tract in the medulla of the brain. These signals inhibit feeding behavior. In a related mechanism, the peptide CCK is released in response to stimulation of the intestines by certain types of food, especially fatty ones. CCK reduces frequency of eating and size of meals. As depicted schematically in FIG. 4, both gastric distension and CCK act synergistically to inhibit feeding behavior.

### VAGAL BLOCKING AND/OR STIMULATION

In commonly assigned disclosures, application Ser. No. 10/079,21 now U.S. Pat. No. ______, and U.S. Pat. No. 6,611,715, pulsed electrical neuromodulation therapy for obesity and other medical conditions is obtained by providing electrical pulses to the vagus nerve(s) via an implanted lead comprising plurality of electrodes. In those disclosures, the electrical pulses are provided by at least one electrode on the lead. This patent application is directed to system and method for neuromodulation of vagal activity, wherein vagal block with or without vagal stimulation may be used to provide therapy for obesity, weight loss, eating disorders, and other gastrointestinal disorders such as FGIDs, gastroparesis, gastro-esophageal reflex disease (GERD), pancreatitis, ileus and the like.

The gastrointestinal tract and central nervous system (CNS) engage each other in two-way communication. This has both parasympathetic and sympathetic components. Of particular interest in this disclosure is the parasympathetic component or the vagal pathway, which is shown in conjunction with FIG. 5.

In some gastrointestinal (GI) disorders, to provide therapy, stimulation of the vagus nerve(s) is adequate and is the preferred mode of providing therapy. For other GI disorders, to provide therapy, stimulation and selective block is the preferred mode of therapy. For some GI disorders, vagal nerve(s) blocking only is the preferred mode of providing therapy. Advantageously, the method and system disclosed in this patent application can provide vagal blocking and/or vagal stimulation to provide therapy for obesity and other gastrointestinal disorders.

As is shown in conjunction with FIG. 6 when vagal pathway is stimulated, the stimulation is conducted both in the Afferent (towards the brain) and Efferent (away from the brain) direction. Shown in conjunction with FIG. 7, by placing blocking electrodes proximal to the stimulating electrodes, and supplying blocking pulses, the conduction in the Afferent direction (towards the brain) can be blocked or significantly reduced. The blocking pulses may be 500 Hz or other frequency, as described later in this disclosure. This is useful for certain GI disorders, for example ileus.

Shown in conjunction with FIG. 8, the blocking electrodes may be placed distal to the stimulating electrodes. If the stimulator provides blocking pulses to the blocking electrode, then the vagus nerve(s) impulses in the Efferent direction are either blocked or are significantly reduced. As the vagus nerves are involved in pancreatitus, the down-regulating vagal activity can used to treat pancreatitus.

It will be clear to one of ordinary skill in the art, that by selectively placing the blocking electrode, selective block can be obtained when the stimulator applies blocking pulses to the blocking electrode. Selective Efferent block is depicted in conjunction with FIG. 9. As shown in the figure, because of the selective placement of blocking electrode(s), only the impulses to visceral organ 2 are blocked or significantly reduced, and impulses to visceral organ-1 and visceral organ-2 continue unimpeded. Selective Afferent block can also be achieved, and is depicted in conjunction with FIG. 10. Here the nerve impulses to visceral organ-4 and visceral organ-5 are selectively blocked. An example would be where Afferent vagal pulses are desired, but impulses to the heart and vocal cords would be blocked. Thus, advantageously providing the desired therapy without the side effects of voice or cardiac complications such as bradycardia.

### BACKGROUND OF NEUROMODULATION

Most nerves in the human body are composed of thousands of fibers of different sizes. This is shown schematically in FIG. 11. The different sizes of nerve fibers, which carry signals to and from the brain, are designated by groups A, B, and C. The vagus nerve, for example, may have approximately 100,000 fibers of the three different types, each carrying signals. Each axon or fiber of that nerve conducts only in one direction, in normal circumstances. In the vagus nerve sensory fibers outnumber parasympathetic fibers four to one.

In a cross section of peripheral nerve it is seen that the diameter of individual fibers vary substantially, as is also shown schematically in FIG. 12. The largest nerve fibers are approximately 20 μm in diameter and are heavily myelinated (i.e., have a myelin sheath, constituting a substance largely composed of fat), whereas the smallest nerve fibers are less than 1 μm in diameter and are unmyelinated.

The diameters of group A and group B fibers include the thickness of the myelin sheaths. Group A is further subdivided into alpha, beta, gamma, and delta fibers in decreasing order of size. There is some overlapping of the diameters of the A, B, and C groups because physiological properties, especially in the form of the action potential, are taken into consideration when defining the groups. The smallest fibers (group C) are unmyelinated and have the slowest conduction rate, whereas the myelinated fibers of group B and group A exhibit rates of conduction that progressively increase with diameter.

Nerve cells have membranes that are composed of lipids and proteins, and have unique properties of excitability such that an adequate disturbance of the cell's resting potential can trigger a sudden change in the membrane conductance. Under resting conditions, the inside of the nerve cell is approximately −90 mV relative to the outside. The electrical signaling capabilities of neurons are based on ionic concentration gradients between the intracellular and extracellular compartments. The cell membrane is a complex of a bilayer of lipid molecules with an assortment of protein molecules embedded in it, separating these two compartments. Electrical balance is provided by concentration gradients which are maintained by a combination of selective permeability characteristics and active pumping mechanism.

A nerve cell can be excited by increasing the electrical charge within the neuron, thus increasing the membrane potential inside the nerve with respect to the surrounding extracellular fluid. The threshold stimulus intensity is the value at which the net inward current (which is largely determined by Sodium ions) is just greater than the net outward current (which is largely carried by Potassium ions), and is typically around −55mV inside the nerve cell relative to the outside (critical firing threshold). If however, the threshold is not reached, the graded depolarization will not generate an action potential and the signal will not be propagated along the axon. This fundamental feature of the nervous system i.e., its ability to generate and conduct electrical impulses, can take the form of action potentials, which are defined as a single electrical impulse passing down an axon. This action potential (nerve impulse or spike) is an “all or nothing” phenomenon, that is to say once the threshold stimulus intensity is reached, an action potential will be generated.

To stimulate an excitable cell, it is only necessary to reduce the transmembrane potential by a critical amount. When the membrane potential is reduced by an amount ΔV, reaching the critical or threshold potential. When the threshold potential is reached, a regenerative process takes place: sodium ions enter the cell, potassium ions exit the cell, and the transmembrane potential falls to zero (depolarizes), reverses slightly, and then recovers or repolarizes to the resting membrane potential (RMP). For a stimulus to be effective in producing an excitation, it must have an abrupt onset, be intense enough, and last long enough.

Cell membranes can be reasonably well represented by a capacitance C, shunted by a resistance R as shown by an electrical model in FIG. 13, where neuronal process is divided into unit lengths, which is represented in an electrical equivalent circuit. Each unit length of the process is a circuit with its own membrane resistance (rₘ), membrane capacitance (cₘ), and axonal resistance (rₐ).

When the stimulation pulse is strong enough, an action potential will be generated and propagated. As shown in FIG. 14, the action potential is traveling from right to left. Immediately after the spike of the action potential there is a refractory period when the neuron is either unexcitable (absolute refractory period) or only activated to sub-maximal responses by supra-threshold stimuli (relative refractory period). The absolute refractory period occurs at the time of maximal Sodium channel inactivation while the relative refractory period occurs at a later time when most of the Na⁺ channels have returned to their resting state by the voltage activated K⁺ current. The refractory period has two important implications for action potential generation and conduction. First, action potentials can be conducted only in one direction, away from the site of its generation, and secondly, they can be generated only up to certain limiting frequencies.

A single electrical impulse passing down an axon is shown schematically in FIG. 15. The top portion of the figure (A) shows conduction over mylinated axon (fiber) and the bottom portion (B) shows conduction over nonmylinated axon (fiber). These electrical signals will travel along the nerve fibers.

The information in the nervous system is coded by frequency of firing rather than the size of the action potential. In terms of electrical conduction, myelinated fibers conduct faster, are typically larger, have very low stimulation thresholds, and exhibit a particular strength-duration curve or respond to a specific pulse width versus amplitude for stimulation, compared to unmyelinated fibers. The A and B fibers can be stimulated with relatively narrow pulse widths, from 50 to 200 microseconds (μs), for example. The A fiber conducts slightly faster than the B fiber and has a slightly lower threshold. The C fibers are very small, conduct electrical signals very slowly, and have high stimulation thresholds typically requiring a wider pulse width (300-1,000 μs) and a higher amplitude for activation. Because of their very slow conduction, C fibers would not be highly responsive to rapid stimulation. Selective stimulation of only A and B fibers is readily accomplished. The requirement of a larger and wider pulse to stimulate the C fibers, however, makes selective stimulation of only C fibers, to the exclusion of the A and B fibers, virtually unachievable inasmuch as the large signal will tend to activate the A and B fibers to some extent as well.

As shown in FIG. 16, when the distal part of a nerve is electrically stimulated, a compound action potential is recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories as shown in the Table one below,

TABLE 1

| | Conduction | Fiber | |
|---|---|---|---|
| Fiber | Velocity | Diameter | |
| Type | (m/sec) | (μm) | Myelination |
| A Fibers | | | |
| Alpha | 70-120 | 12-20 | Yes |
| Beta | 40-70 | 5-12 | Yes |
| Gamma | 10-50 | 3-6 | Yes |
| Delta | 6-30 | 2-5 | Yes |
| B Fibers | 5-15 | <3 | Yes |
| C Fibers | 0.5-2.0 | 0.4-1.2 | No |

Vagus nerve blocking and stimulation, performed by the system and method of the current patent application, is a means of directly affecting central function, as well as, peripheral function. FIG. 17 shows cranial nerves have both afferent pathway 19 (inward conducting nerve fibers which convey impulses toward the brain) and efferent pathway 21 (outward conducting nerve fibers which convey impulses to an effector). Vagus nerve (the 10^{th} cranial nerve) is composed of 80% afferent sensory fibers carrying information to the brain from the head, neck, thorax, and abdomen. The sensory afferent cell bodies of the vagus reside in the nodose ganglion and relay information to the nucleus tractus solitarius (NTS).

The vagus nerve spans from the brain stem all the way to the splenic flexure of the colon. Not only is the vagus the parasympathetic nerve to the thoracic and abdominal viscera, it also the largest visceral sensory (afferent) nerve. Sensory fibers outnumber parasympathetic fibers four to one. In the medulla, the vagal fibers are connected to the nucleus of the tractus solitarius (viceral sensory), and three other nuclei. The central projections terminate largely in the nucleus of the solitary tract, which sends fibers to various regions of the brain (e.g., the thalamus, hypothalamus and amygdala).

### PRIOR ART

Prior art is generally directed to adapting cardiac pacemaker technology for nerve stimulation, where U.S. Pat. No. 5,263,480 (Wernicke et al.) and Pat. No. 5,188,104 (Wemicke et al.) are generally directed to treatment of eating disorders with vagus nerve stimulation using an implantable neurocybernetic prosthesis (NCP), which is a “cardiac pacemaker-like” device. There is no disclosure for vagal blocking.

U.S. Pat. No. 5,540,730 (Terry et al.) is generally directed to treating motility disorders with vagus nerve stimulation using an implantable neurocybernetic prosthesis (NCP), which is a “cardiac pacemaker-like” device.

U.S. Pat. No. 6,600,954 B2 (Cohen et al.) is generally directed to selectively blocking propagation of body-generated action potentials particularly useful for pain control.

U.S. Pat. No. 6,684,105 B2 (Cohen et al.) is generally directed to an apparatus for unidirectional nerve stimulation.

### SUMMARY OF THE INVENTION

The current patent disclosure overcomes many of the shortcomings of the prior art by externalizing the stimulator for nerve blocking and/or nerve stimulation. The energy used for supplying blocking and stimulating pulses can be a significant drain on the battery of an implanted pulse generator, since the blocking pulses may be higher frequency relative to the stimulating pulses. In the method and system of this invention, the external stimulator is adapted to be inductively coupled to an implanted stimulus-receiver to provide power and data. This system is advantageous because it eliminates repeated surgeries that are required for conventional implanted pulse generators that are surgically replaced at the end of their service life. A further advantage of the system and method of the current disclosure is that the external stimulator (with an optional telemetry module) can be remotely interrogated and programmed over the internet. This eliminates the need for patient to visit physicians office or clinic every time the device needs to be re-programmed.

It is an object of the invention to provide a method and system for providing electrical pulses for nerve blocking with or without selective electrical stimulation of vagus nerve(s) of a patient, with an external stimulator system which is inductively coupled to an implanted stimulus-receiver with electrodes adapted to be in contact with the nerve tissue to be stimulated or blocked.

It is another object of the invention, that the electrical pulses are provided to vagus nerve(s) or its branches or part thereof.

It is another object of the invention to provide electrical pulses for nerve blocking and/or nerve stimulation for treating, controlling or alleviating the symptoms for at least one of obesity, motility disorders, eating disorders, inducing weight loss, FGIDs, gastroparesis, gastro-esophageal reflex disease (GERD), pancreatitis, and ileus.

It is another object of the invention to modulate portions of the vagus nerve(s) to provide therapy for obesity or to induce weight loss

It is another object of the invention to selectively block vagus nerve(s) or its branches or part thereof.

It is another object of the invention to store predetermined programs comprising electrical pulse parameters, in the memory of the external stimulator.

It is another object of the invention to provide a method of providing electrical pulses to the vagus nerve(s) of a patient for treating obesity or inducing weight loss utilizing an external stimulator in conjunction with an implanted unit comprising high value capacitor(s) for storing energy.

It is another object of the invention, that the combination of external component and implanted component comprise proximity sensing means for alignment of external (primary) coil and implanted (secondary) coil.

It is another object of the invention to provide feedback regulation for pulses provided to secondary (implanted) coil by primary (external) coil.

It is another object of the invention to provide an optional telemetry module for the external stimulator for communication and data exchange remotely, over a wide area network.

It is yet another aspect of the invention, that the external stimulator may be interrogated and programmed remotely.

These and other objects are provided by one or more of the embodiments described below.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram depicting vagal nerves in a patient.

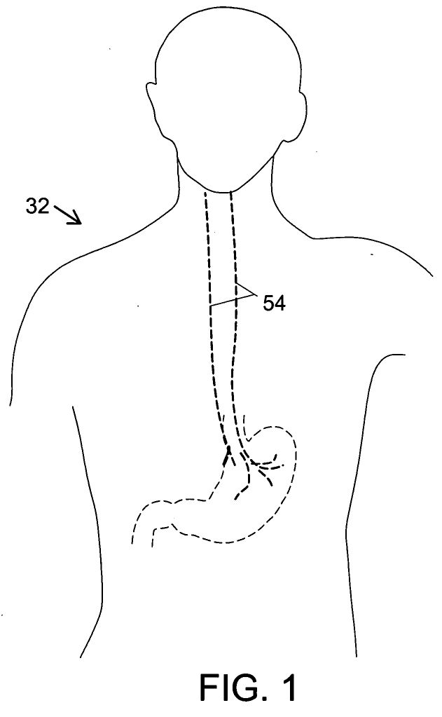

FIG. 2 is a diagram showing vagal nerve innervation to the viceral organs.

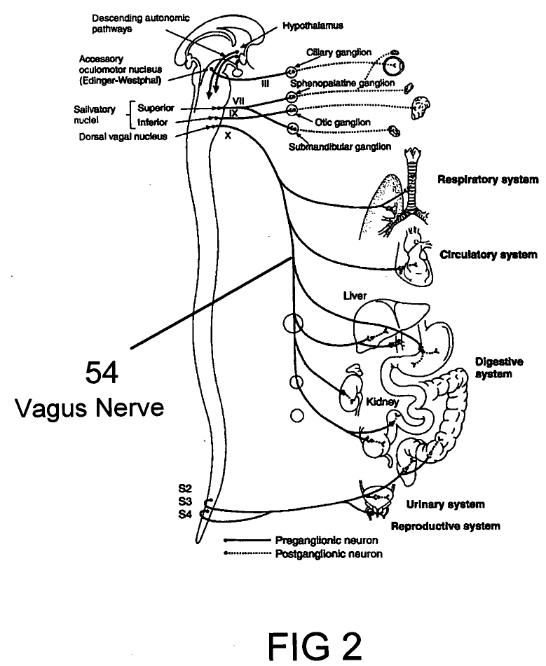

FIG. 3 is a schematic diagram showing the relationship of meals and satiety signals.

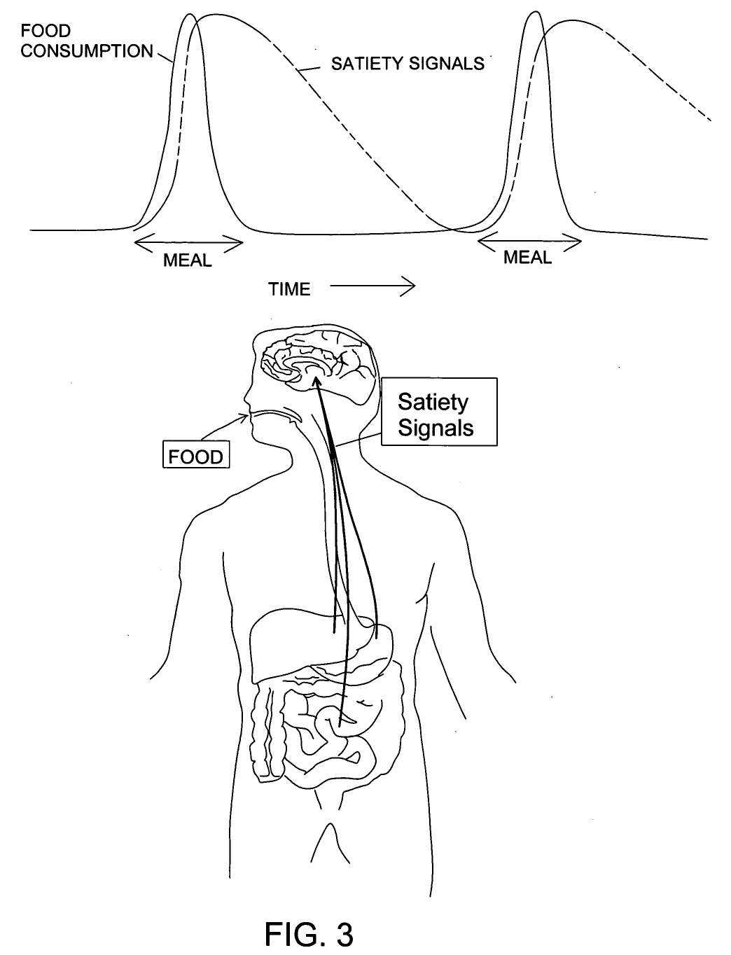

FIG. 4 is a schematic diagram showing impulses traveling via the vagus nerve in response to gastric distention and CCK release.

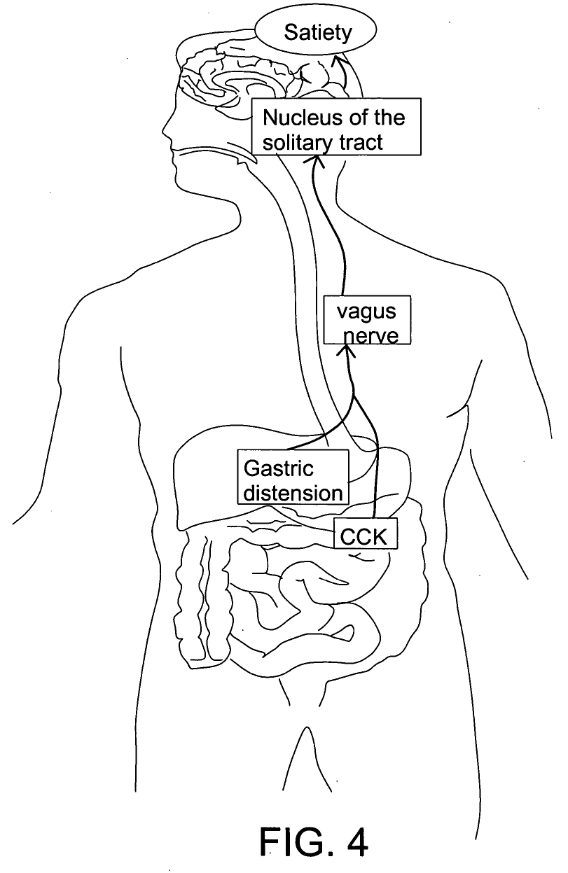

FIG. 5 is a diagram depicting two-way communication between the gut and central nervous system (CNS).

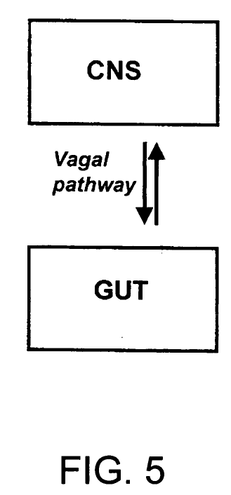

FIG. 6 is a diagram showing conduction of nerve impulses in both afferent and efferent direction with artificial electrical stimulation.

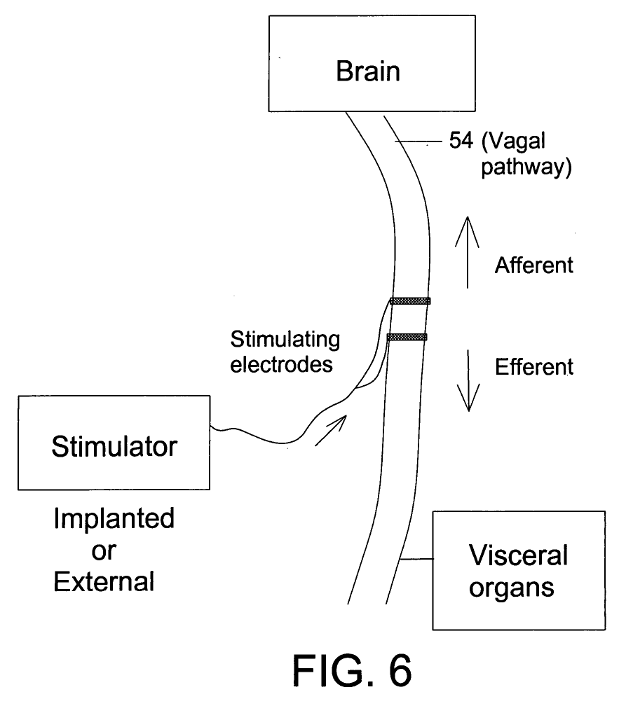

FIG. 7 is a diagram depicting blocking in the afferent direction, but conducting in the efferent direction with electrical stimulation.

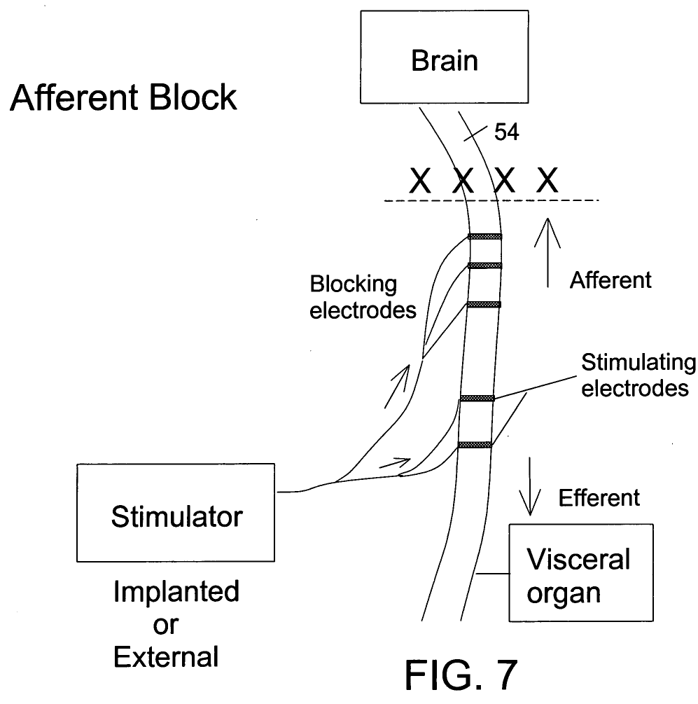

FIG. 8 is a diagram depicting electrical stimulation with conduction in the afferent direction and blocking in the efferent direction.

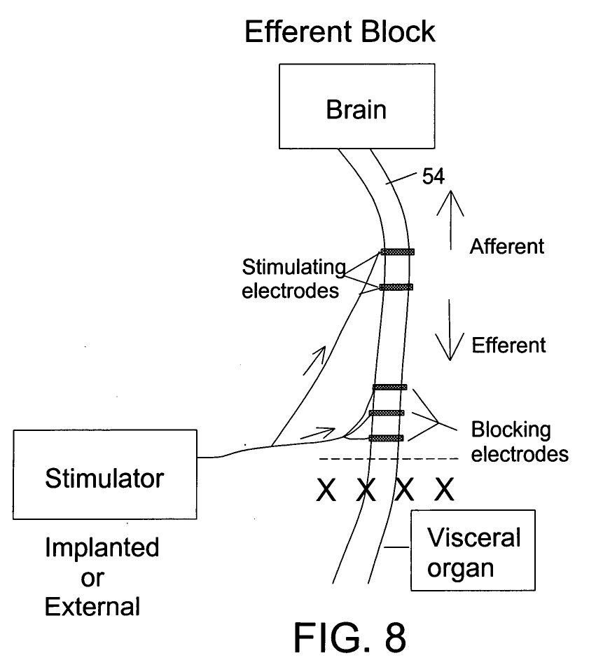

FIG. 9 is a diagram depicting electrical stimulation with conduction in the afferent direction and selective organ blocking in the efferent direction.

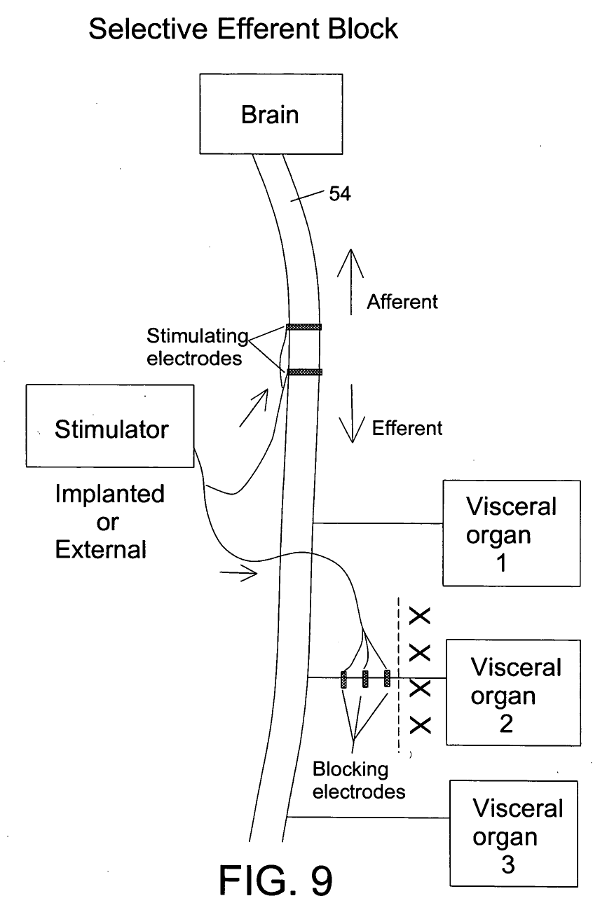

FIG. 10 is a diagram depicting electrical stimulation with conduction in the efferent direction and selective organ blocking in the afferent direction.

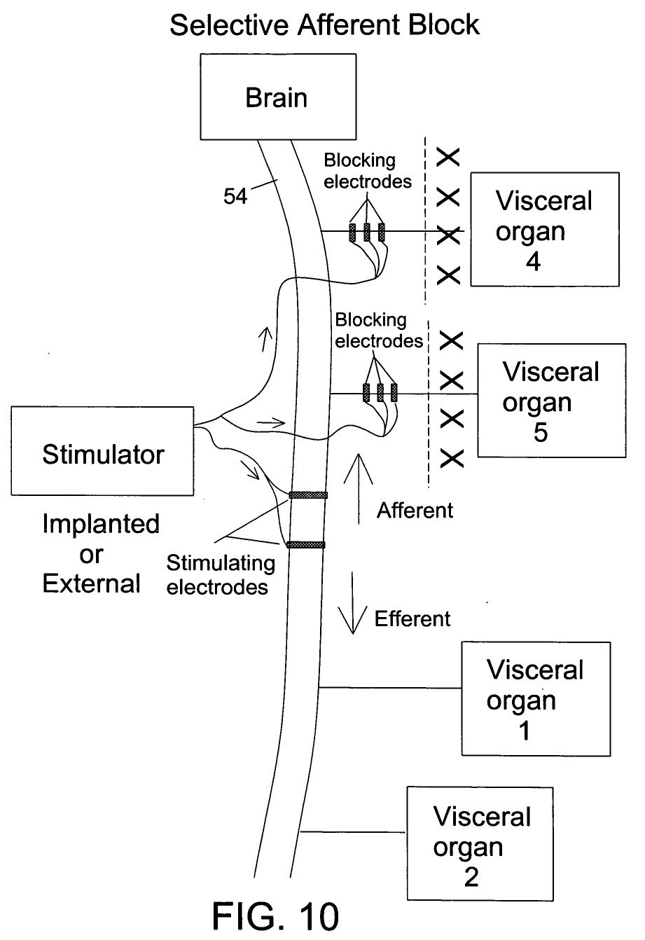

FIG. 11 is a diagram of the structure of a nerve.

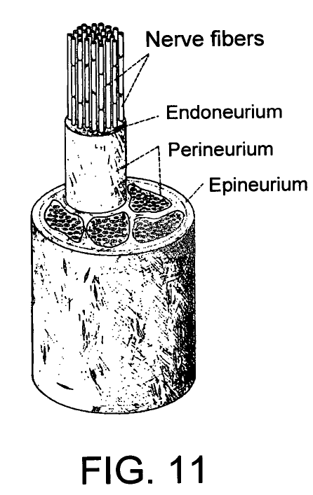

FIG. 12 is a diagram showing different types of nerve fibers.

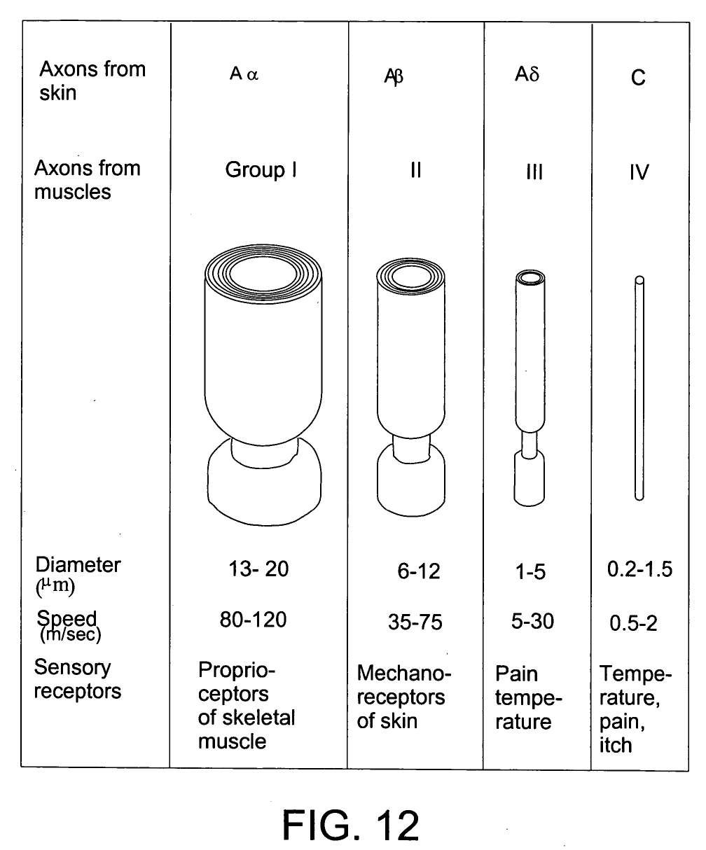

FIG. 13 is a schematic illustration of electrical circuit model of nerve cell membrane.

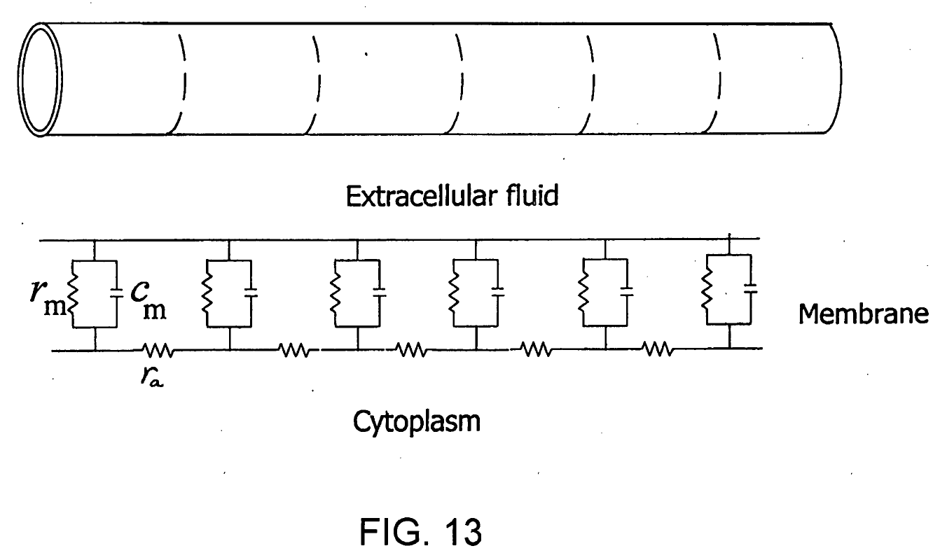

FIG. 14 is an illustration of propagation of action potential in nerve cell membrane.

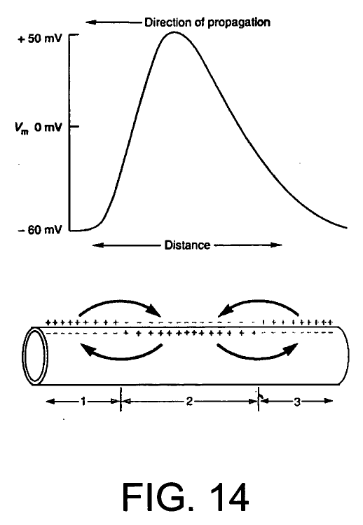

FIG. 15 is an illustration showing propagation of action potential along a myelinated axon and non-myelinated axon.

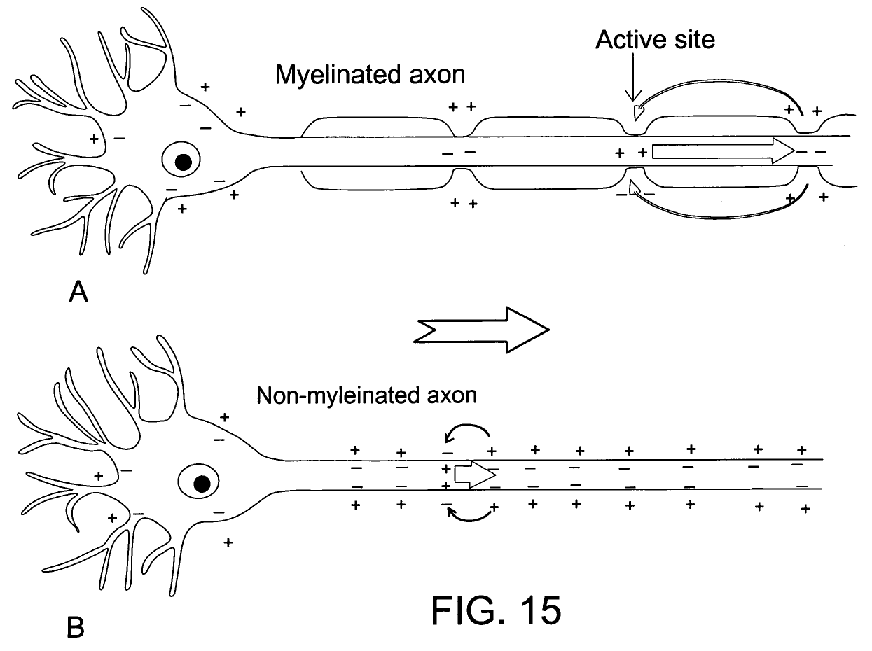

FIG. 16 is a diagram showing recordings of compound action potentials.

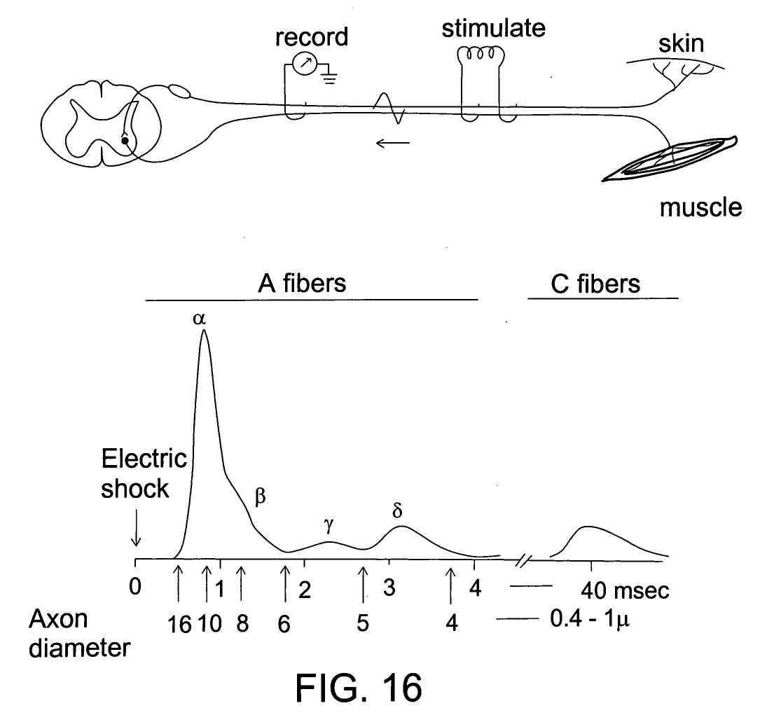

FIG. 17 is a schematic diagram of brain showing afferent and efferent pathways.

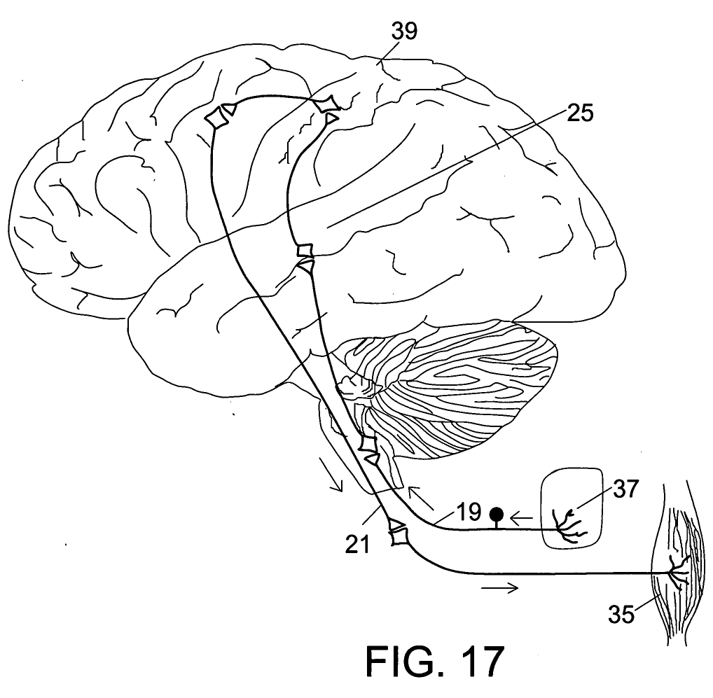

FIG. 18 is a diagram of implanted components of stimulation/blocking system with multiple electrodes around anterior and posterior vagal nerves.

FIG. 19 is a diagram showing the implanted components, and an external stimulator coupled to an implanted stimulus-receiver.

FIG. 20 is a diagram showing placement of the external (primary) coil in relation of the implanted stimulus-receiver.

FIG. 21 is a diagram showing the relative placement of the two coils (primary and secondary).

FIG. 22 is a simplified block diagram depicting supplying amplitude and pulse width modulated electromagnetic pulses to an implanted coil.

FIG. 23 shows coupling of the external stimulator and the implanted stimulus-receiver.

FIG. 24 is a schematic of the passive circuitry in the implanted stimulus-receiver.

FIG. 25 is a schematic of an alternative embodiment of the implanted stimulus-receiver.

FIG. 26 is another alternative embodiment of the implanted stimulus-receiver.

FIG. 27 depicts an external stimulator adapted to couple to an implanted stimulus-receiver.

FIG. 28 is an overall block diagram of the components of the external stimulator.

FIG. 29 is a block diagram of programmable array logic interfaced to the programming station.

FIG. 30 is a block diagram showing details of programmable logic array unit.

FIG. 31 is a diagram showing details of the interface between the programmable array logic and interface unit.

FIG. 32 is a diagram showing the circuitry of a pulse generator

FIG. 33 is a schematic diagram showing the implantable lead and one form of stimulus-receiver.

FIG. 34 is a schematic block diagram showing a system for neuromodulation of nerve tissue, with an implanted component which is both RF coupled and contains a capacitor power source.

FIG. 35 is a schematic diagram of the pulse generator and two-way communication through a server.

FIG. 36 is a diagram depicting wireless remote interrogation and programming of the external pulse generator.

FIG. 37 is a schematic diagram of the wireless protocol.

FIG. 38 is a simplified block diagram of the networking interface board.

FIGS. 39 and 40 are simplified diagrams showing communication of modified PDA/phone with an external stimulator via a cellular tower/base station.

### DESCRIPTION OF THE INVENTION

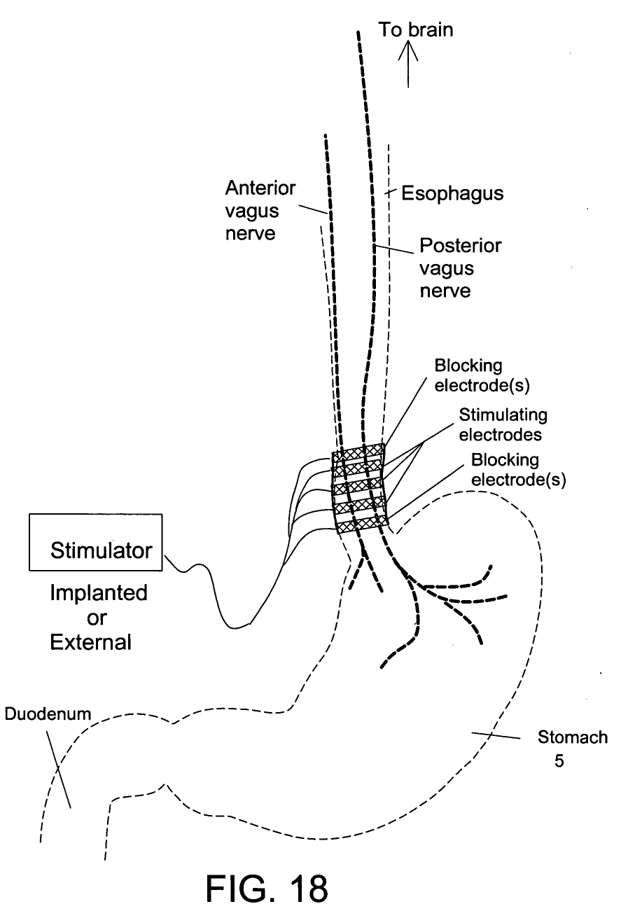

To provide vagal blocking and/or vagal stimulation therapy to a patient, blocking and stimulation electrodes are implanted at the appropriate sites. In one preferred embodiment, without limitation, multiple electrodes comprising both blocking and stimulation electrodes are placed in a band. As shown in conjunction with FIG. 18, the band comprising multiple electrodes is wrapped around the esophagus, close to the junction of esophagus and the stomach 5 (just below the diaphragm). Alternatively, the individual electrodes do not have to be in a band, and may be individual electrodes, connected to the body of the lead via insulated conductors. In such a case, the portion of the electrode contacting the nerve tissue would be exposed and the rest of the electrode being insulated with a non-conductive material such as silicone or polyurethane. Such electrodes are well known in the art.

The electrodes may be implanted using laproscopic surgery or alternatively a surgical exposure may be made for implantation of the electrodes at the appropriate site to be stimulated and/or blocked. After placing the electrodes, the terminal portion of the lead is tunneled to a subcutaneous site where the electronics package is to be implanted. The terminal end of the lead is connected to the stimulus-receiver or the implanted stimulator. The patient is surgically closed, and electrical pulse delivery can begin once the patient has fully recovered from the surgery.

In the method and system of this invention, stimulation without block may be provided. Additionally, stimulation with selective block may be provided. Furthermore, block alone (without stimulation) may be provided, which would be functionally equivalent to reversible vagotomy.

Blocking of nerve impulses, unidirectional blocking, and selective blocking of nerve impulses is well known in the scientific literature. Some of the general literature is listed below and is incorporated herein by reference. (a) “Generation of unidirectionally propagating action potentials using a monopolar electrode cuff”, Annals of Biomedical Engineering, volume 14, pp. 437-450, By Ira J. Ungar et al. (b) “An asymmetric two electrode cuff for generation of unidirectionally propagated action potentials”, IEEE Transactions on Biomedical Engineering, volume BME-33, No. 6, June 1986, By James D. Sweeney, et al. (c) A spiral nerve cuff electrode for peripheral nerve stimulation, IEEE Transactions on Biomedical Engineering, volume 35, No. 11, November 1988, By Gregory G. Naples. et al. (d) “A nerve cuff technique for selective excitation of peripheral nerve trunk regions, IEEE Transactions on Biomedical Engineering, volume 37, No. 7, July 1990, By James D. Sweeney, et al. (e) “Generation of unidirectionally propagated action potentials in a peripheral nerve by brief stimuli”, Science, volume 206 pp.1311-1312, Dec. 14, 1979, By Van Den Honert et al. (f) “A technique for collision block of perpheral nerve: Frequency dependence” IEEE Transactions on Biomedical Engineering, MP-12, volume 28, pp. 379-382, 1981, By Van Den Honert et al. (g) “A nerve cuff design for the selective activation and blocking of myelinated nerve fibers” Ann. Conf. of the IEEE Engineering in Medicine and Biology Soc., volume 13, No. 2, p 906, 1991, By D. M Fitzpatrick et al. (h) “Orderly recruitment of motoneurons in an acute rabbit model”, “Ann. Conf. of the IEEE Engineering in Medicine and Biology Soc., volume 20, No. 5, page 2564, 1998, By N. J. M. Rijkhof, et al. (i) “Orderly stimulation of skeletal muscle motor units with tripolar nerve cuff electrode”, IEEE Transactions on Biomedical Engineering, volume 36, No. 8, pp. 836, 1989, By R. Bratta. (j) M. Devor, “Pain Networks”, Handbook of Brand Theory and Neural Networks, Ed. M. A. Arbib, MIT Press, page 698, 1998.

Blocking can be generally divided into 3 categories: (a) DC or anodal block, (b) Wedenski Block, and (c) Collision block. In anodal block there is a steady potential which is applied to the nerve causing a reversible and selective block. In Wedenski Block the nerve is stimulated at a high rate causing the rapid depletion of the neurotransmitter. In collision blocking, unidirectional action potentials are generated anti-dromically. The maximal frequency for complete block is the reciprocal of the refractory period plus the transit time, i.e. typically less than a few hundred hertz. The use of any of these blocking techniques can be applied for the practice of this invention, and all are considered within the scope of this invention.

For supplying the electrical signals, two embodiments are disclosed for carrying out the invention. In the first embodiment the implanted stimulus-receiver is a simpler device comprising an implanted secondary coil along with the associated circuitry. In the second embodiment, the implanted unit also comprises a high value capacitor (supercap) for storing charge for up to a few hours. In both embodiments, the initial power is supplied from an external unit via an external (primary) coil.

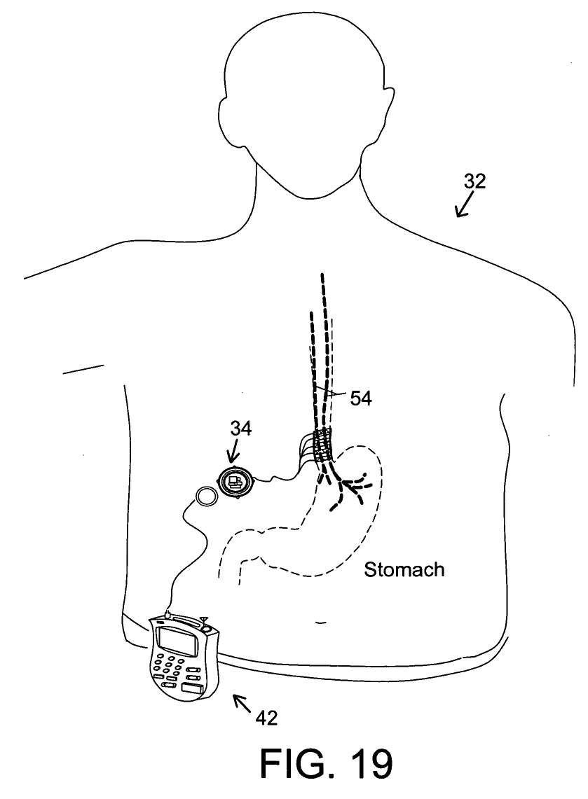

In the first embodiment, for therapy to commence, the primary (external) coil 46 is placed on the skin 60 on top of the surgically implanted (secondary) coil 48. An adhesive tape may be placed on the skin 60 and external coil 46 such that the external coil 46, is taped to the skin 60. Alternatively, a special garment may be used for the placement of the primary (external) coil, or other means may be used. Shown in conjunction with FIGS. 19, 20 and 21, the external stimulator 42 is adapted to inductively couple with an implanted stimulus-receiver 34. The primary (external) coil 46 of the external pulse generator 42 inductively transfers pulses to the implanted stimulus-receiver 34, which has multiple electrodes (via the lead) in contact with the appropriate nerve tissue for vagal blocking and/or stimulation.

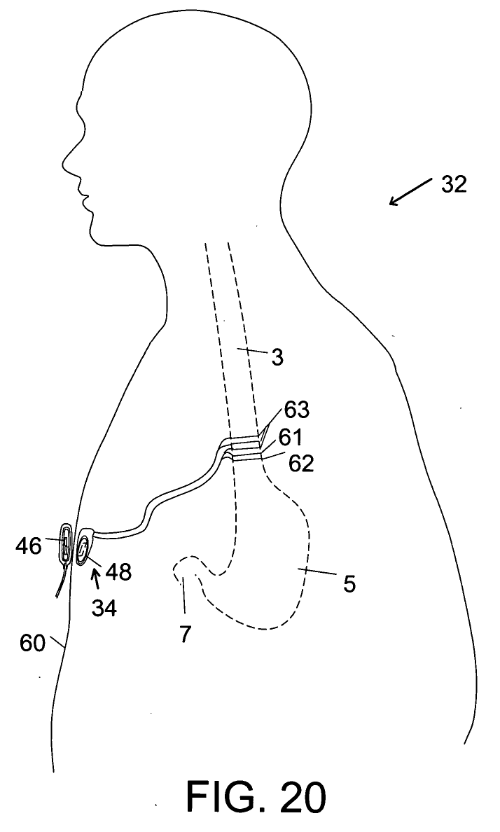

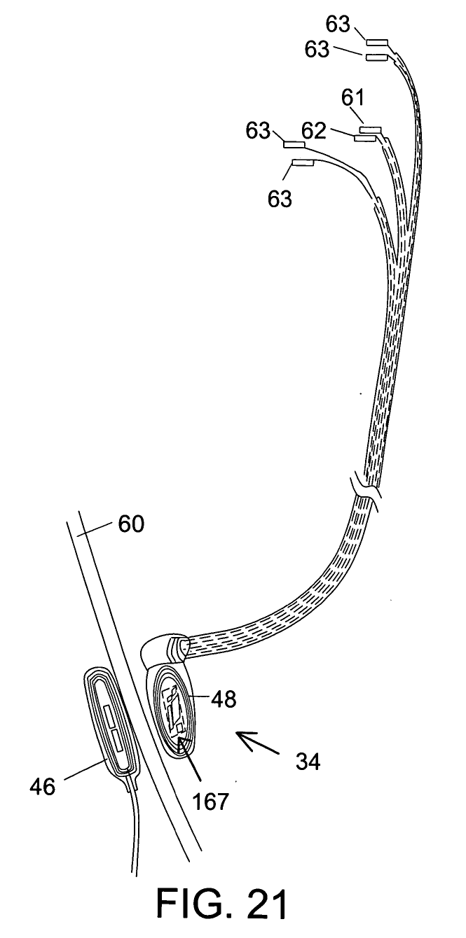

When the two coils, which are the primary coil 46 (external) and secondary coil 48 (implanted) are arranged with their axes on the same line, current sent through coil 46 creates a magnetic field that cuts coil 48 which is subcutaneous. Consequently, a voltage will be induced in the secondary coil 48 whenever the field strength of the primary coil 46 is changing. This induced voltage is similar to the voltage of self-induction but since it appears in the second coil because of current flowing in the first, it is a mutual effect and results from the mutual inductance between the two coils (primary 46 and secondary 48). The degree of coupling of these two coils depends upon the physical spacing between the coils and how they are placed with respect to each other. Maximum coupling exists when they have a common axis and are as close together as possible (but separated by skin 60). The coupling is least when the coils are far apart or are placed so their axes are at right angles. As depicted in FIGS. 20 and 21, the secondary coil 48 inside the stimulus-receiver 34 is approximately along the same axis as the primary coil 46.

FIG. 21 shows a schematic diagram of an implantable stimulus-receiver 34. The stimulus-receiver receives the pulses from outside the body. The proximal end of the stimulus-receiver 34 comprises the secondary coil 48 and electronic circuitry (hybrid) 167 which is hermetically sealed, and covered with silicone. It may have one or more anchoring sleeve(s) 130 for tying it to the subcutaneous tissue.

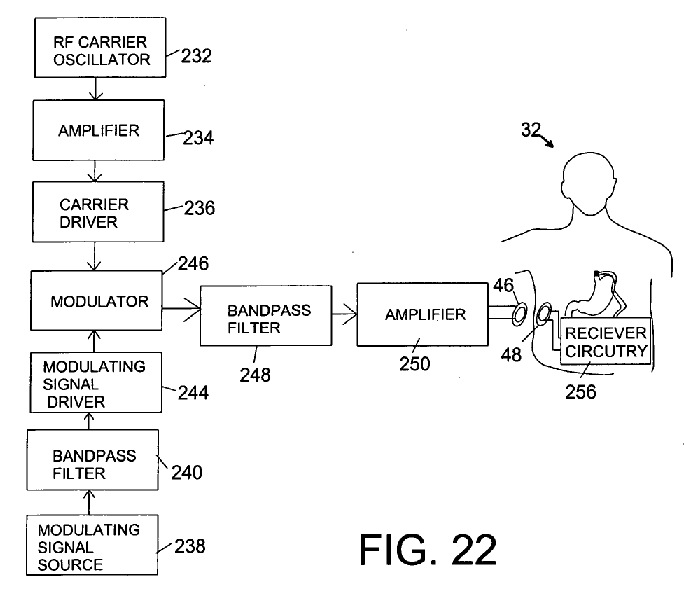

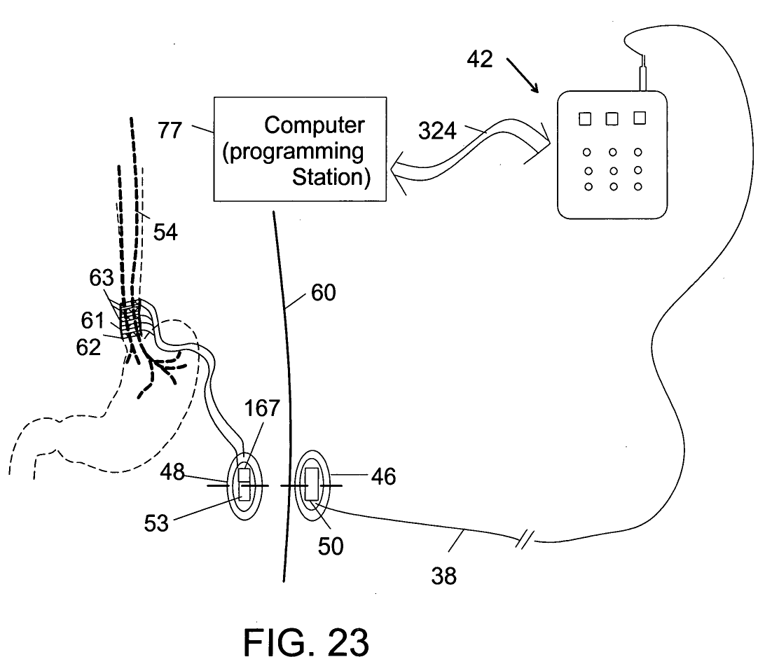

FIG. 22 shows in block diagram form, the delivery methodology to deliver vagal blocking and/or stimulation pulses. A modulator 246 receives analog (sine wave) high frequency “carrier” signal and modulating signal. The modulating signal can be multilevel digital, binary, or even an analog signal. In this embodiment, mostly multilevel digital type i.e., pulse amplitude and pulse width modulated signals are used. The modulated signals are conditioned 248, amplified 250, and transmitted via a primary coil 46 which is external to the body. Shown in conjunction with FIG. 23, a secondary coil 48 of the implanted stimulus-receiver, receives, demodulates, and delivers these pulses to the vagal tissue 54. The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

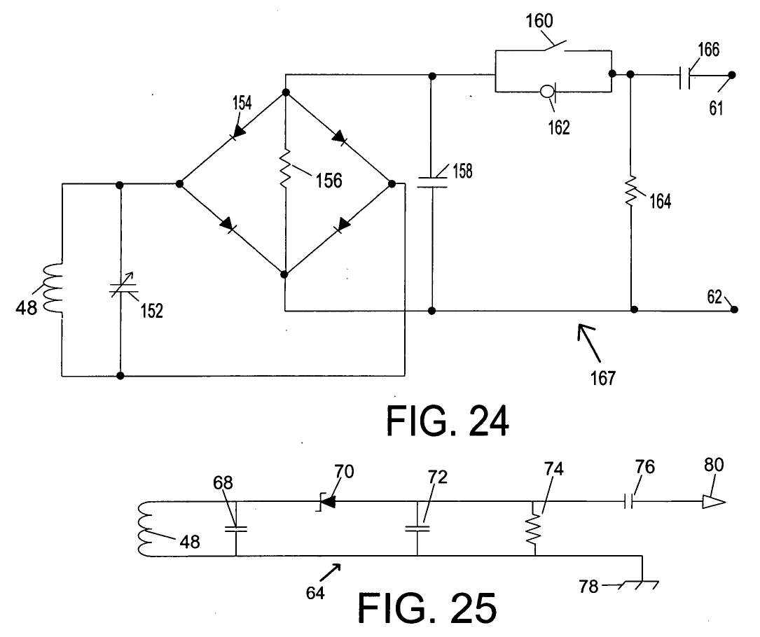

The implantable stimulus-receiver 34 has circuitry at the proximal end, and has stimulating and blocking electrodes at the distal end of the lead. The circuitry contained in the proximal end of the implantable stimulus-receiver 34 is shown schematically in FIG. 24, for one embodiment. In this embodiment, the circuit uses all passive components. Approximately 25 turn copper wire of 30 gauge, or comparable thickness, is used for the primary coil 46 and secondary coil 48. Other functional equivalents may also be used. This wire is concentrically wound with the windings all in one plane. The frequency of the pulse-waveform delivered to the implanted coil 48 can vary, and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. For stimulation/blocking signals, the return path of signals from cathode 61 will be through anode 62 placed in proximity to the cathode 61 for “Bipolar” stimulation. In this embodiment bipolar mode of stimulation is used, however, the return path can be connected to the remote ground connection (case) of implantable circuit 167, providing for much larger intermediate tissue for “Unipolar” stimulation. The “Bipolar” stimulation offers localized stimulation of tissue compared to “Unipolar” stimulation and is therefore, preferred in this embodiment. The implanted circuit 167 in this embodiment is passive, so a battery does not have to be implanted.

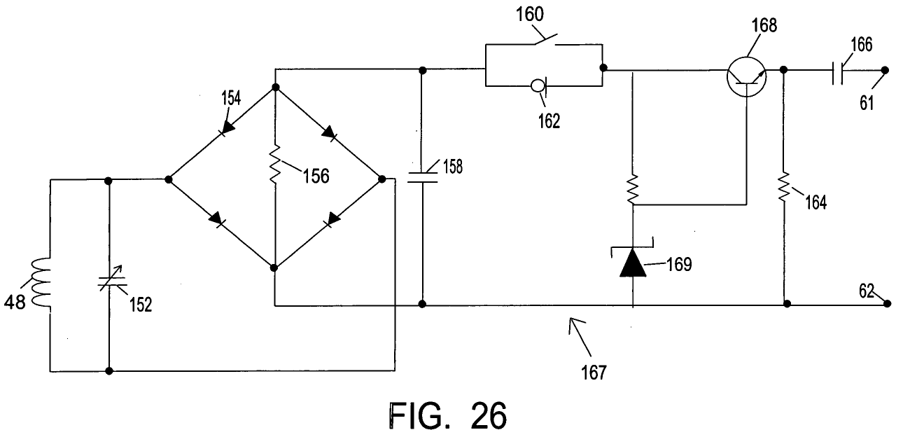

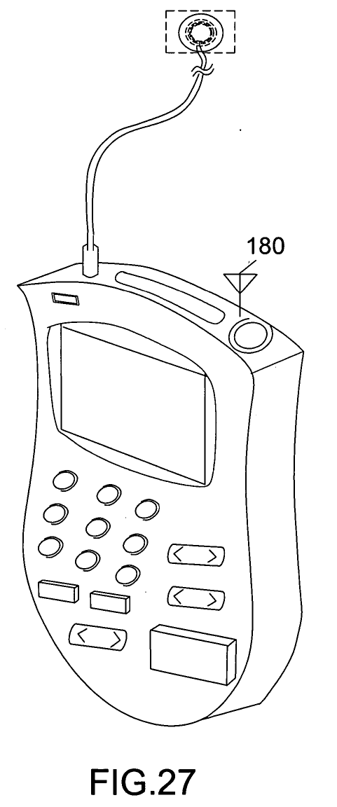

The circuitry shown in FIGS. 25 and 26 can be used as an alternative for the implanted stimulus-receiver 34. The circuitry of FIG. 25 is a slightly simpler version, and circuitry of FIG. 26 contains a conventional NPN transistor 168 connected in an emitter-follower configuration.

For efficient energy transfer to occur, it is important that the primary (external) 46 and secondary (implanted) coils 48 be positioned along the same axis and be optimally positioned relative to each other. In this embodiment, the external coil 46 may be connected to proximity sensing circuitry 50. The correct positioning of the external coil 46 with respect to the implanted coil 48 is indicated by turning “on” of a light emitting diode (LED) on the external stimulator 42.

Many different forms of proximity sensing mechanisms may be used. In one embodiment optimal placement of the external (primary) coil 46 may be done with the aid of proximity sensing circuitry incorporated in the system. Proximity sensing occurs utilizing a combination of external and implantable components. The implanted components contains a relatively small magnet composed of materials that exhibit Giant Magneto-Resistor (GMR) characteristics such as Samarium-cobalt, a coil, and passive circuitry. As was depicted in conjunction with FIG. 23, the external coil 46 and proximity sensor circuitry 50 are rigidly connected in a convenient enclosure which is attached externally on the skin. The sensors measure the direction of the field applied from the magnet to sensors within a specific range of field strength magnitude. The dual sensors exhibit accurate sensing under relatively large separation between the sensor and the target magnet. As the external coil 46 placement is “fine tuned”, the condition where the external (primary) coil 46 comes in optimal position, i.e. is located adjacent and parallel to the subcutaneous (secondary) coil 48, along its axis, is recorded and indicated by a light emitting diode (LED) on the external stimulator 42. Other forms of proximity sensing mechanisms may also be used

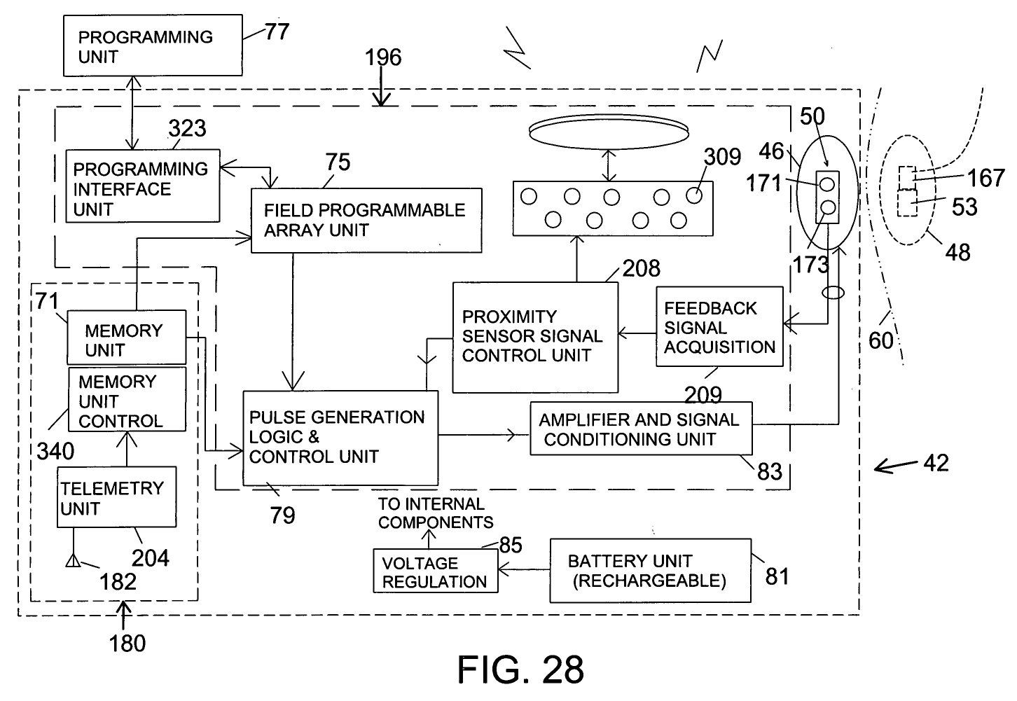

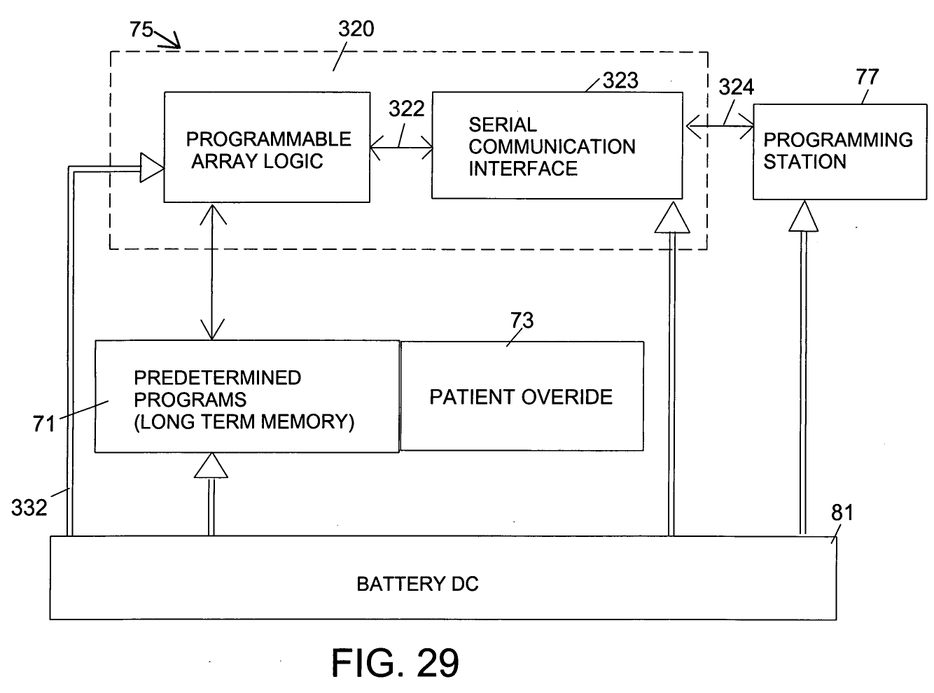

FIG. 27 shows a front view of one embodiment of an external stimulator 42. The external stimulator 42 contains the circuitry, rechargeable power source, external coil and, an optional telemetry module. FIG. 28 shows a block diagram of the external stimulator 42. The pre-packaged or “customized” programs are stored in the memory unit 71. This represents memory with a readable and writeable portion and a non-volatile pre-programmable portion. A Field Programmable Array Unit (FPGA) 75 and a random access component (RAM) 320 and Random addressable storage logic 340, facilitates application of logic to edit and change the “current” parameters being utilized for pulse generation. The programmable unit interface 323 provides an interface to a programming unit (portable computer system) 77, which allows re-loading of a new set of programs. The pulse generation component 79 generates pulses of well-defined parameters, selected from the programmed parameters that exist in the memory unit 71. The pulse signal generation unit 79 provides its signal to be amplified and conditioned at the amplifier and signal conditioning unit 83 which then provides these signals to the primary (external) inductive coil 46. The logic and control unit can provide both the blocking and stimulating pulses.

In one embodiment a pair of sensors 174 senses the position of the implanted magnet 53 and the sensor signal is fed back to the proximity sensor control block 208 via the feedback signal conditioning unit 209. The feedback signal provides a proportional signal for modification of the frequency, amplitude and pulse-width of the pulse being generated by the pulse signal generator unit 79. The sensor unit has two sensors 171, 173 that sense the location of the implanted magnet 53. The implanted (secondary) coil 48 is rigidly connected to the passive circuit and magnet 53. The skin 60 separates the subcutaneous and external components. The external components are placed on the skin, with the primary coil 46 in close proximity and optimally situated with respect to the implanted (secondary) coil 48.

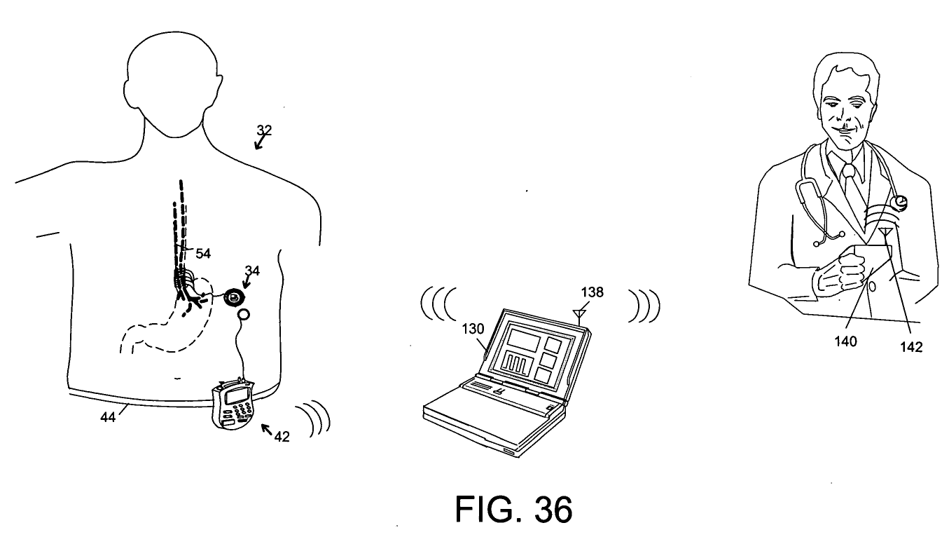

As is shown in conjunction with FIG. 28 and FIG. 35, the external pulse generator 42 is composed of three modules or sub-assemblies. The first sub-assembly is the pulse generation and signal conditioning components 196, the second is the battery 81, and the third is the telemetry and memory unit 180. The presently preferred embodiment, comprises proximity sensing and feedback circuitry. This invention can be practiced without the proximity sensing and feedback circuitry. The pulse generator is able to function as supplier of electric pulses to the nerve tissue without the proximity feedback loop and the telemetry module. These modules or sub-assemblies also provide for a scalable external pulse generator 42. In the telemetry module, a wireless antenna 182 provides a means of communication to the external pulse generator 42 and the wireless remote server 189. A programming unit 77 can also be physically connected to the external stimulator 42 (via the Programming Unit Interface 323) in a tethered manner for loading of new programs or changing parameters of an existing program.

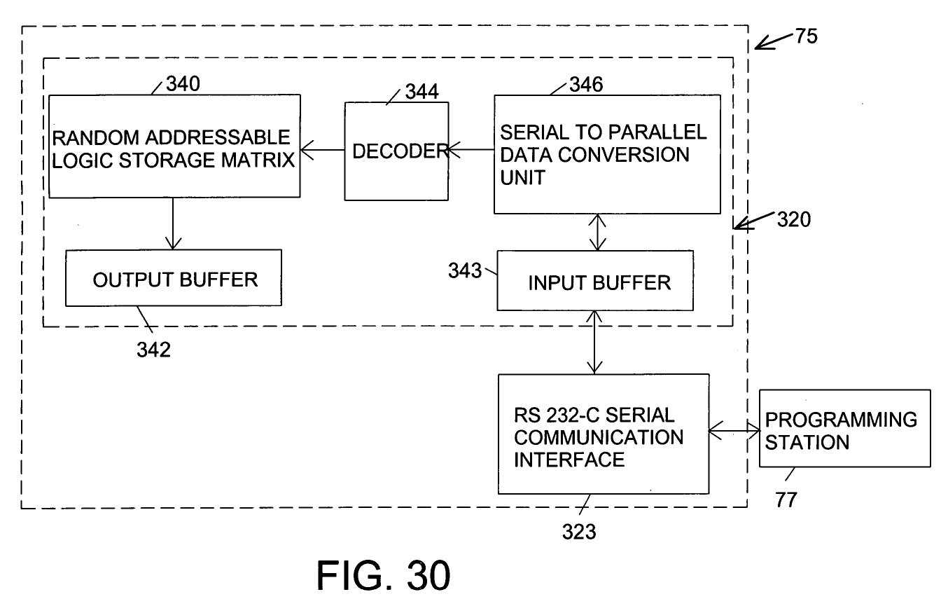

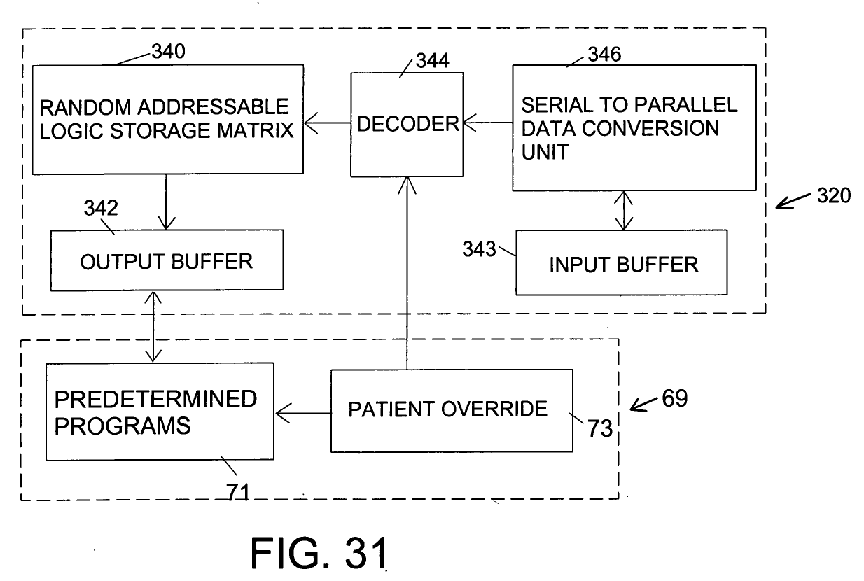

FIG. 29 shows the Programmable Array Logic and Interface Unit 75 interfaced to the Programming Station 77. The programming station allows the user to change the program parameters for various stimulation and/or blocking programs. The programming station is connected to the Programmable Array Unit 75 with an RS232-C serial connection 324. The main purpose of the serial line interface is to provide an RS232-C standard interface. This method enables any portable computer with a serial interface to communicate and program the parameters for storing the various programs. The serial communication interface 323 receives the serial data, buffers this data and converts it to a 16 bit parallel data. The Programmable Array Logic 320 component of Programmable Array Unit 75 receives the parallel data bus and stores or modifies the data into a random access matrix. This array of data also contains special logic and instructions along with the actual data. These special instructions also provide an algorithm for storing, updating and retrieving the parameters from long-term memory. The Programmable Array Unit 320, interfaces with Long Term Memory 71 to store the pre-determined programs. All the previously modified programs can be stored here for access at any time. The programs will consist of specific parameters and each unique program will be stored sequentially in Long Term Memory 71. A battery unit 81 provides power to all the components shown above. The logic for the storage and decoding is stored in the Random Addressable Storage Matrix (RASM) 340 (FIG. 30).

FIG. 30 shows greater details for the Programmable Logic Array Unit 320. The Input Buffer block 343 stores the serial data in temporary register storage. This accumulation allows for the serial to parallel conversion to occur. The serial to 16 bit parallel block sets up 16 bits of data 346, as created from the RS232-C serial data. This parallel data bus will communicate the data and the address information. The decoder block 344 decodes address information for the Random Addressable Logic Storage Matrix 340 from which to access the data i.e. programmer parameters. The Output Buffer 342 provides an interface to the Long Term Memory 71.

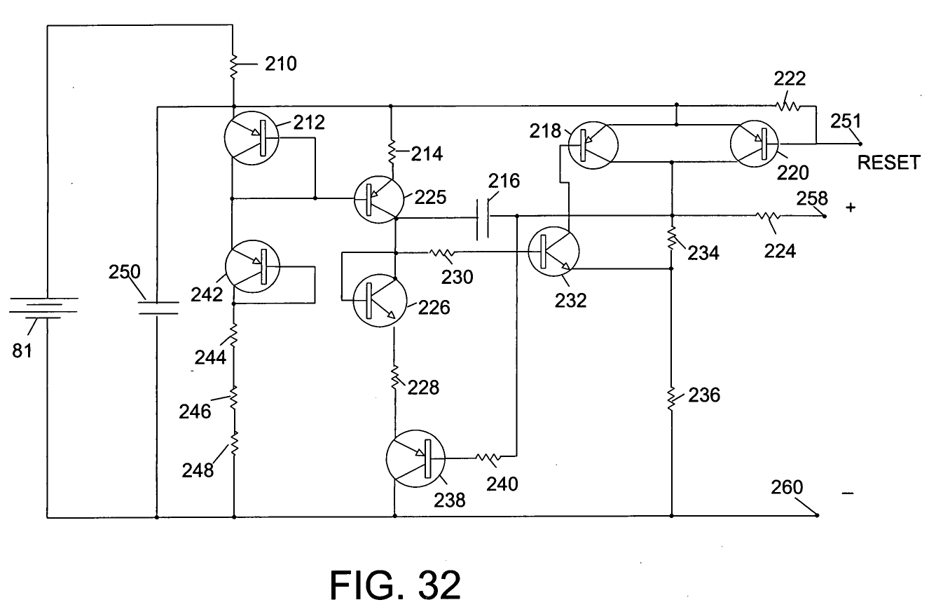

FIG. 31 shows schematically the details of the interface between the Programmable Array Logic 320 and Interface Unit 75 which is connected to the Predetermined Programs block (Long Term Memory) 71. The Patient Override 73 is essentially a control scheme for initializing or starting a program at any intermediate point. The Field Programmable array provides a reconfigurable mechanism to store data and associated instructions for the programs. It supports adding, modifying or retrieving the data from a Random Addressable Logic Storage Matrix 340. This is also a scheme for treating “flexible” logic description and control. It is flexible by providing the ability to reprogram and even redesign existing programs previously installed programs. As was shown schematically in FIG. 28, the health care provider can load and reload stimulation programs of choice. This allows the authorized user to create, modify and select for execution, programs to use for a particular time period.

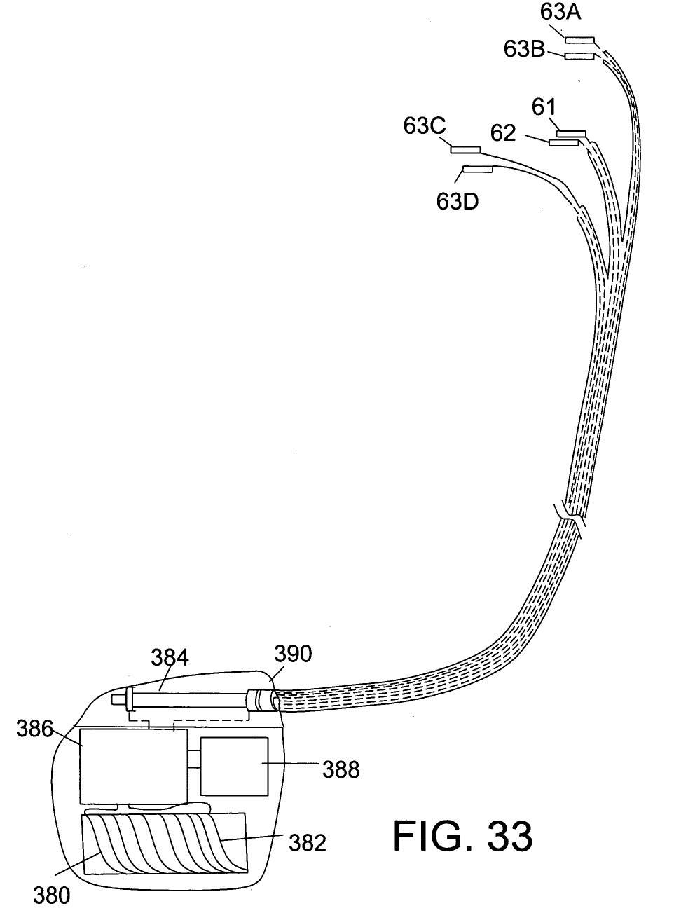

FIG. 32 shows an example of pulse generator circuitry, which exhibits typical multivibrator functionality. It will be clear to one skilled in the art, that other circuits can also be used. This circuit produces regularly occurring pulses where the amplitude, pulse width and frequency is adjustable. The battery 81 is the main external power source for this circuit. The capacitor 250 is connected in parallel with the battery 81. The combination of transistors 212, 242 and 225, and resistors 210, 244, 246 and 248 acts as a constant current source generated at the collector of transistor 226. The transistor 212 has collector connected to the emitter of transistor 242 and base of transistor 225. The transistors 212 and 242 are connected to provide a constant voltage drop. Likewise, transistor 226 also acts as a diode with a resistor 228 connected in series and further connected to the negative terminal of the line at terminal 260. Capacitor 216 provides timing characteristics and its value helps determine pulse width and pulse frequency. The output of the oscillator appears at terminal 258.

Initially, the capacitor 216 gets charged with current from the path of resistor 234 and 236 while all the transistors are turned off. As the capacitor charges up transistor 232 will become forward biased and current will flow via resistors 230 and 236 from the base to emitter resistors. This action turns on the transistor 218 and the positive voltage from the power supply 81 is made available at the base of transistor 238 through resistor 240. This results in the transistor 238 getting turned on. The conduction of transistor 238 causes capacitor 216 to discharge. The time constant for the charge and discharge of capacitor 216 is determined by value of the resistors 228 and 240 and capacitor 216. After the time constant, transistor 232 turns off, and this in turn turns off transistors 238 and 218. A reset mechanism for this multivibrator can be provided by setting a positive voltage, for example 2.5 volts, to the base of transistor 220. This positive increase in voltage turns on transistor 220 followed by transistor 238. The turning on of transistor 238 discharges the capacitor 216 and the reset operation is complete.

Conventional integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver these pre-determined programs is well known to those skilled in the art.

The selective blocking and/or stimulation to the vagal nerve tissue can be performed by “pre-determined” programs or by “customized” programs, where the electrical parameters are selectively programmed, for specific therapy to the individual patient. The electrical parameters which can be individually programmed, include variables such as pulse amplitude, pulse width, frequency of stimulation, type of pulse (e.g. blocking pulses may be sinusoidal), stimulation on-time, and stimulation off-time. Table two below defines the approximate range of parameters, Table 2--Electrical parameter range delivered to the nerve for stimulation and/or blocking

TABLE 2
Electrical parameter range
delivered to the nerve for stimulation and/or blocking

| PARAMER | RANGE |
|---|---|
| Pulse Amplitude | 0.1 Volt-10 Volts |
| Pulse width | 20 μS-5 mSec. |
| Stim. Frequency | 5 Hz-200 Hz |
| Freq. for blocking | DC to 5,000 Hz |
| On-time | 5 Secs-24 hours |
| Off-time | 5 Secs-24 hours |

The parameters in Table 2 are the electrical signals delivered to the nerve tissue via the two stimulation electrodes 61, 62 (or blocking electrodes) at the nerve tissue. It being understood that the signals generated by the external pulse generator 42 and transmitted via the primary coil 46 are larger, because the attenuation factor between the primary coil and secondary coil is approximately 10-20 times, depending upon the distance, and orientation between the two coils. Accordingly, the range of transmitted signals of the external pulse generator 42 are approximately 10-20 times larger than shown in Table 2.

The implanted lead component of the system is somewhat similar to cardiac pacemaker leads, except for distal portion 40 (or electrode end) of the lead. The lead terminal preferably is linear, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The stimulation electrodes 61, 62 (or blocking electrodes) are typically implanted adjacent to the nerve tissue to be stimulated or blocked.

The electrodes may be made of pure platinum, platinum/Iridium alloy or platinum/iridium coated with titanium nitride. The conductor connecting the terminal to the electrodes is made of an alloy of nickel-cobalt. The implanted lead design variables are also summarized in table three below.

TABLE 3
Lead design variables

| | | | Conductor | | |
|---|---|---|---|---|---|
| Proximal | | | (connecting | | Distal |
| End | Lead body- | | proximal | | End |
| Lead | Insulation | | and distal | Electrode - | Electrode - |
| Terminal | Materials | Lead-Coating | ends) | Material | Type |
| Linear | Polyurethane | Antimicrobial | Alloy of | Pure | Wrap-around |
| bipolar | | coating | Nickel- | Platinum | electrodes |
| | | | Cobalt | | |
| Bifurcated | Silicone | Anti- | | Platinum- | Standard Ball |
| | | Inflammatory | | Iridium | and Ring |
| | | coating | | (Pt/Ir) Alloy | electrodes |
| | Silicone with | Lubricious | | Pt/Ir coated | Steroid |
| | Polytetrafluoroethylene | coating | | with Titanium | eluting |
| | (PTFE) | | | Nitride | |
| | | | | Carbon | |

Once the lead is fabricated, coating such as anti-microbial, anti-inflammatory, or lubricious coating may be applied to the body of the lead.

### Implanted Stimulus-Receiver Comprising a High Value Capacitor for Storing Charge, Used in Conjunction with an External Stimulator

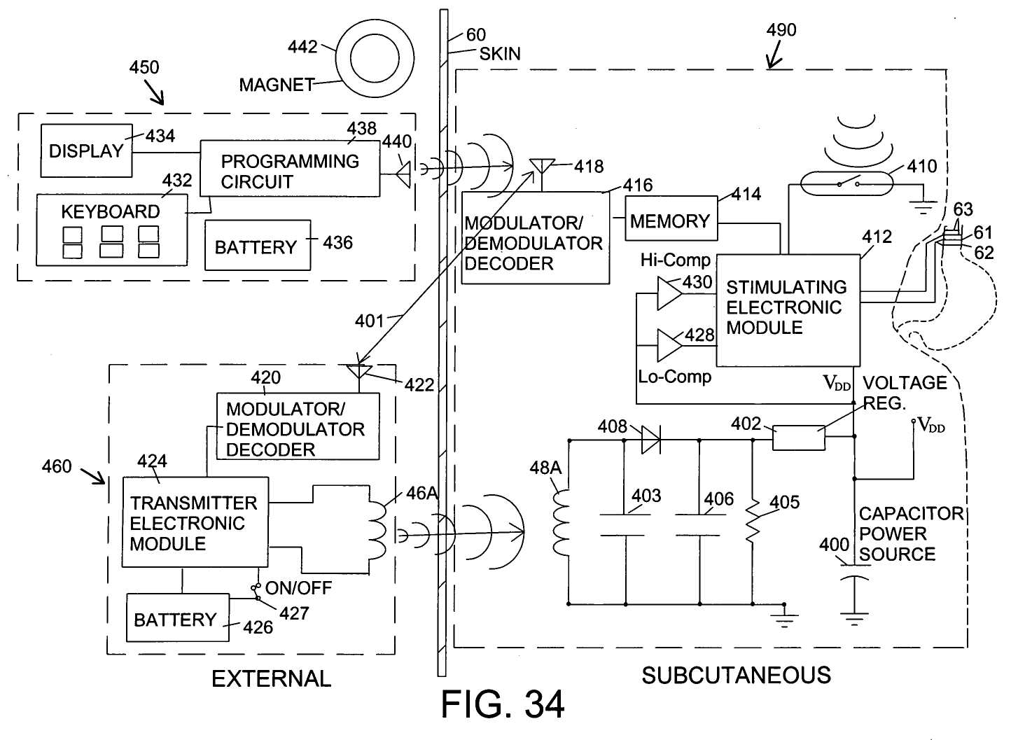

In one embodiment, the implanted stimulus-receiver may be a system which is RF coupled combined with a power source. In this embodiment, the implanted stimulus-receiver also comprises high value, small sized capacitor(s) for storing charge and delivering electric stimulation and/or blocking pulses for up to several hours by itself, once the capacitors are charged. The packaging is shown in FIG. 33. Using mostly hybrid components and appropriate packaging, the implanted portion of the system described below is conducive to miniaturization. As shown in FIG. 33, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment.

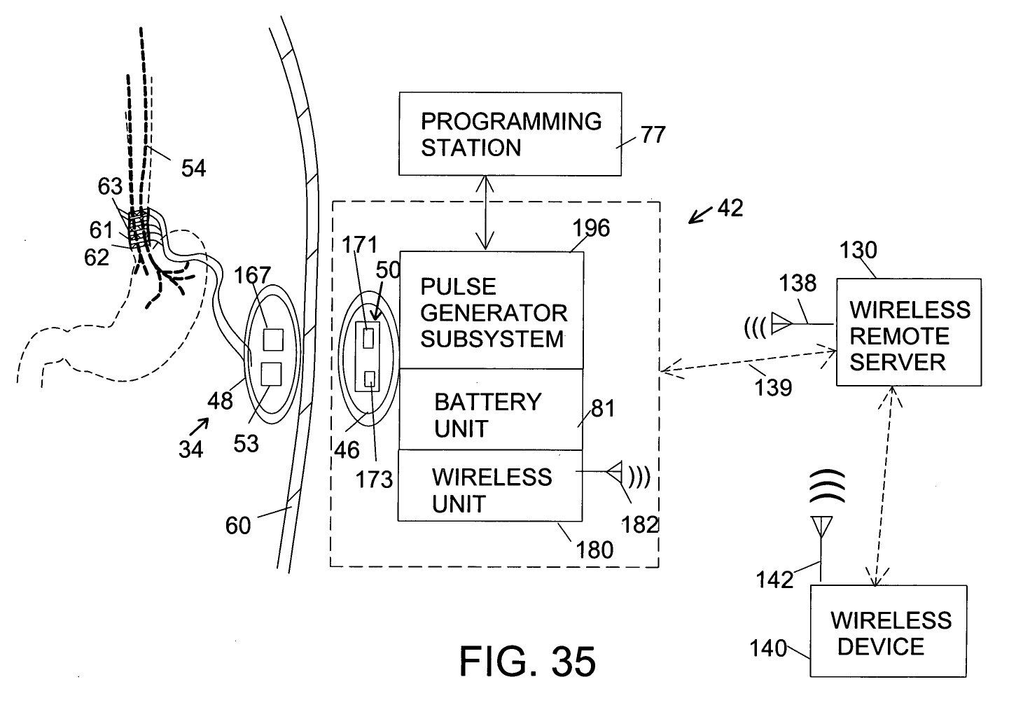

Shown in conjunction with FIG. 34 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641 (available from Pansonic corporation).

The refresh-recharge transmitter unit 460 includes a primary battery 426, an ON/Off switch 427, a transmitter electronic module 442, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 424 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is VDD reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation/blocking by waving the magnet 442 once near the implant. The magnet emits a magnetic force Lₘ which pulls reed switch 410 closed. Upon closure of reed switch 410, stimulation/blocking electronic module 412 in conjunction with memory 414 begins the delivery (or cessation as the case may be) of controlled electronic stimulation/blocking pulses to the nerve tissues via the implanted electrodes. In another mode (AUTO), the stimulation/blocking is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulation/blocking electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

It will be clear from the above disclosure that the implanted stimulus-receiver may be purely a passive device or may comprise a power source, such as a high value capacitor. In either case, initially the energy provided via an external stimulator 42.

### Telemetry Module

Shown in conjunction with FIG. 35, in one embodiment of the invention the external pulse generator 42 has two-way wireless communication capabilities with a remote server, using a communication protocol such as the wireless application protocol (WAP). The purpose of the telemetry module is to enable the physician to remotely, via the wireless medium change the programs, activate, or disengage programs. Additionally, schedules of therapy programs, can be remotely transmitted and verified. Advantageously, the physician is thus able to remotely control the stimulation therapy.

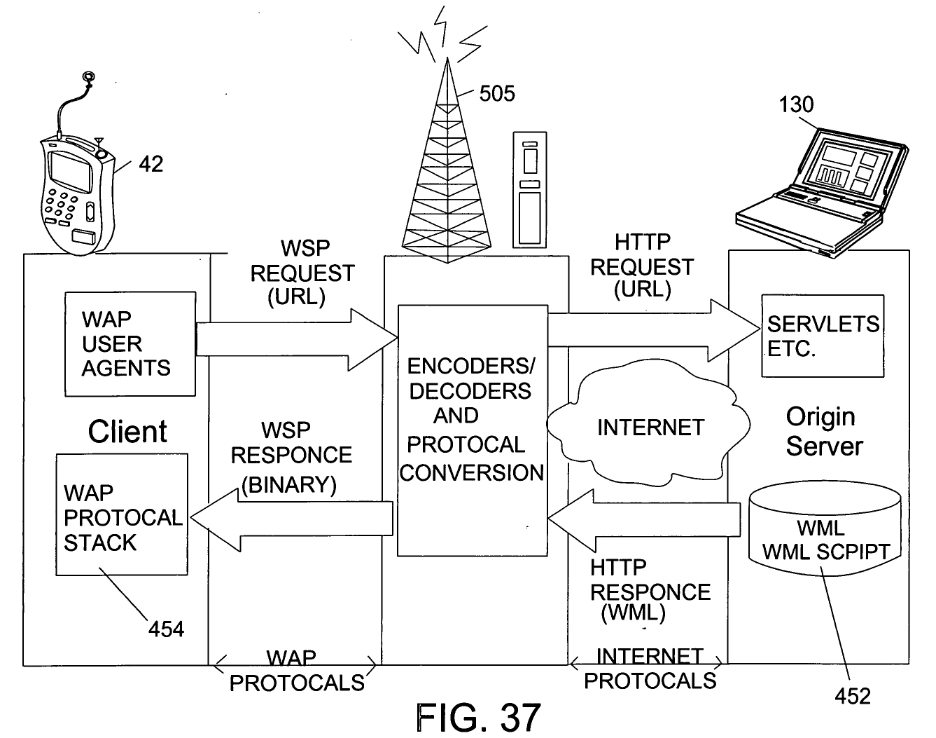

FIG. 36 is a simplified schematic showing the communication aspects between the pulse generator 42 and the remote hand-held computer. A desktop or laptop computer can be a server 130 which is situated remotely, perhaps at a health-care provider's facility or a hospital. The data can be viewed at this facility or reviewed remotely by medical personnel on a wireless internet supported hand-held device 140, which could be a personal data assistant (PDA), for example, a “palm-pilot” from PALM corp. (Santa Clara, Calif.), a “Visor” from Handspring Corp. (Mountain view, Calif.) or on a personal computer (PC) available from numerous vendors or a cell phone or a handheld device being a combination thereof. The physician or appropriate medical personnel, is able to interrogate the external stimulator 42 device and know what the device is currently programmed to, as well as, get a graphical display of the pulse train. The wireless communication with the remote server 130 and hand-held device (wireless internet supported) 140 can be achieved in all geographical locations within and outside the United States (US) that provides cell phone voice and data communication service. The pulse generation parameter data can also be viewed on the handheld devices 140.

The telecommunications component of this invention uses Wireless Application Protocol (WAP). WAP is a set of communication protocols standardizing Internet access for wireless devices. Previously, manufacturers used different technologies to get Internet on hand-held devices. With WAP, devices and services inter-operate. WAP promotes convergence of wireless data and the Internet. The WAP Layers are Wireless Application Envirnment (WAEW), Wireless Session Layer (WSL), Wireless Transport Layer Security (WTLS) and Wireless Transport Layer (WTP).

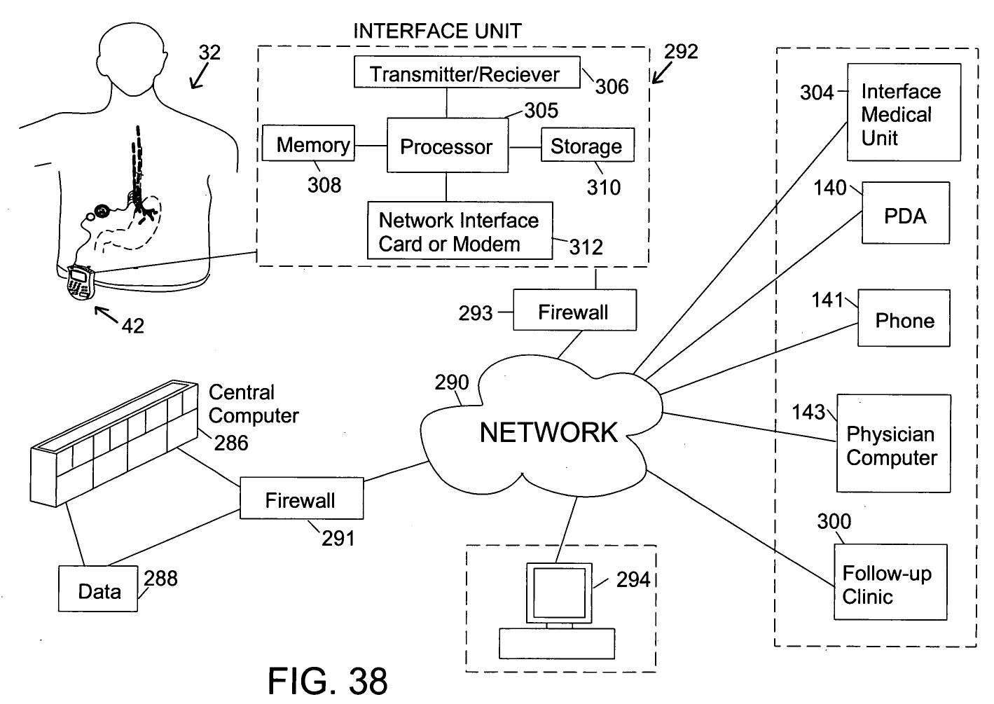

The WAP programming model, which is heavily based on the existing Internet programming model, is shown schematically in FIG. 37. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops. Such features are facilitated with WAP.

The key components of the WAP technology, as shown in FIG. 37, includes 1) Wireless Mark-up Language (WML) 400 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 402 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handles asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications, and well known to those skilled in the art.

The presently preferred embodiment utilizes WAP, because WAP has the following advantages, 1) WAP protocol uses less than one-half the number of packets that the standard HTTP or TCP/IP Internet stack uses to deliver the same content. 2) Addressing the limited resources of the terminal, the browser, and the lightweight protocol stack are designed to make small claims on CPU and ROM. 3) Binary encoding of WML and SMLScript helps keep the RAM as small as possible. And, 4) Keeping the bearer utilization low takes account of the limited battery power of the terminal.

In this embodiment two modes of communication are possible. In the first, the server initiates an upload of the actual parameters being applied to the patient, receives these from the stimulator, and stores these in its memory, accessible to the authorized user as a dedicated content driven web page. The web page is managed with adequate security and password protection. The physician or authorized user can make alterations to the actual parameters, as available on the server, and then initiate a communication session with the stimulator device to download these parameters.

The physician is also able to set up long-term schedules of stimulation therapy for their patient population, through wireless communication with the server. The server in turn communicates these programs to the neurostimulator. Each schedule is securely maintained on the server, and is editable by the physician and can get uploaded to the patient's stimulator device at a scheduled time. Thus, therapy can be customized for each individual patient. Each device issued to a patient has a unique identification key in order to guarantee secure communication between the wireless server 130 and stimulator device 42.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

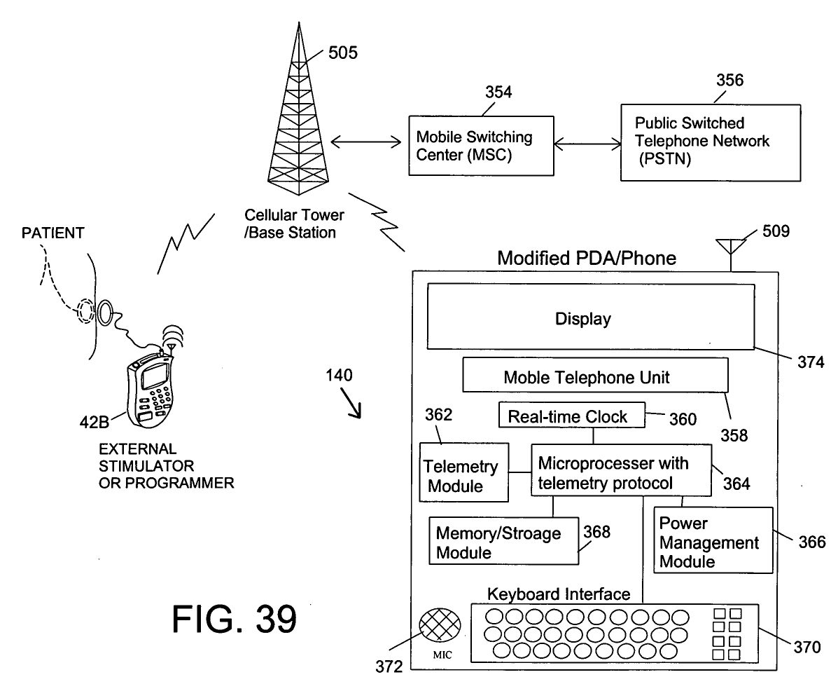

Shown in conjunction with FIG. 38, in one embodiment, the external stimulator 42 and/or the programmer 85 may also be networked to a central collaboration computer 286 as well as other devices such as a remote computer 294, PDA 140, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

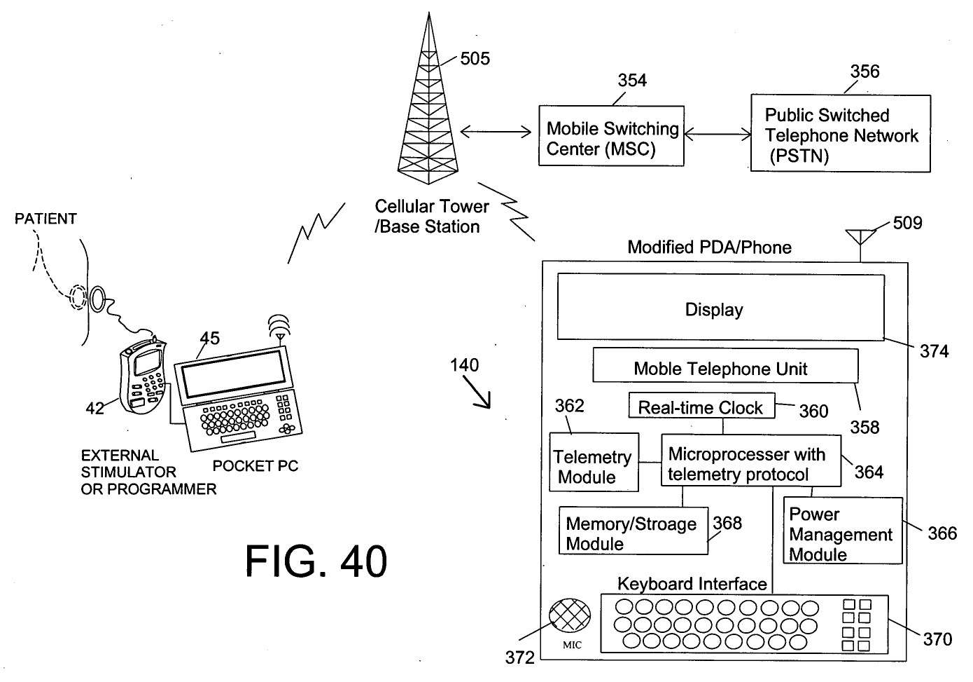

Shown in conjunction with FIG. 36 the physician's remote communication's module is a Modified PDA/Phone 140 in this embodiment. The Modified PDA/Phone 140 is a microprocessor based device as shown in a simplified block diagram in FIGS. 39 and 40. The PDA/Phone 140 is configured to accept PCM/CIA cards specially configured to fulfill the role of communication module 292 of the present invention. The Modified PDA/Phone 140 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 140 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 140 and external stimulator 42. The intent of this invention is to use 3G technology for wireless communication and data exchange, even though in some cases 2.5G is being used currently.

For the system of the current invention, the use of any of the “3G” technologies for communication for the Modified PDA/Phone 140, is considered within the scope of the invention. Further, it will be evident to one of ordinary skill in the art that as future 4G systems, which will include new technologies such as improved modulation and smart antennas, can be easily incorporated into the system and method of current invention, and are also considered within the scope of the invention.

1. A method of providing electrical pulses for nerve blocking, with or without selective electrical stimulation of vagus nerve(s) or its branches or part thereof, comprising the steps of:
providing an external stimulator to generate and transmit electrical pulses, comprising a controller, circuitry to generate blocking and stimulating electrical pulses, and an external coil;
providing an implanted stimulus-receiver means to inductively receive said electrical pulses, comprising circuitry and secondary coil; and
providing a lead with at least two electrodes adapted to be in contact with said nerve(s) or its branches or part thereof in a patient, and in electrical contact with said stimulus-receiver means.

2. The method of claim 1, wherein said nerve blocking is provided for treating, controlling or alleviating the symptoms for at least one of obesity, motility disorders, eating disorders, inducing weight loss, FGIDs, gastroparesis, gastro-esophageal reflex disease (GERD), pancreatitis, and ileus.

3. A method of claim 1, wherein at least one electrode is providing said electric pulses for said nerve blocking.

4. A method of claim 1, wherein said nerve blocking is at least one of afferent blocking, efferent blocking, or organ block.

5. A method of claim 1, wherein said nerve blocking comprises at least one from a group consisting of: DC or anodal block, Wedenski block, and Collision block.

6. A method of claim 1, wherein said nerve blocking is provided to at least one of the left or/and right vagus nerve(s) their branch(es) or part thereof, at one or more sites.

7. The method of claim 1, wherein said external stimulator further comprises means for manually controlling and/or changing said electrical pulses.

8. The method of claim 1, wherein said implanted stimulus receiver means further comprises:
a) means for temporary power storage, and
b) said temporary power storage means is high value capacitors.

9. The method of claim 1, wherein said external stimulator comprises telemetry means for communication and data exchange over a wide area network to remotely interrogate and/or remotely program said external stimulator.

10. The method of claim 1, wherein a proximity sensing means is used to position said external coil with respect to said secondary coil.

11. The method of claim 1, wherein said implanted stimulus-receiver means comprises means for regulating said electric pulses that are provided to said nerve.

12. The method of claim 1, wherein said electrical pulses have predetermined parameter values of the variable components of said electric pulses comprising at least one of pulse amplitude, pulse width, pulse frequency, and on-off timing sequence such that, set(s) of said predetermined parameter values of the variable components of said electric pulses can be stored in memory of said external stimulator as program(s).

13. A method of providing electrical pulses for vagal blocking with or without selective vagal stimulation for treating or alleviating the symptoms for at least one of obesity, eating disorders, inducing weight loss, FGIDs, gastroparesis, gastro-esophageal reflex disease (GERD), pancreatitis, and ileus, comprising the steps of:
providing an external stimulator to generate and transmit electrical pulses, comprising a controller, circuitry to generate blocking and stimulating electrical pulses, and an external coil;
providing an implanted stimulus-receiver means to inductively receive said electrical pulses, comprising circuitry and secondary coil; and
providing a lead with at least two electrodes adapted to be in contact with said nerve(s) or branches or part thereof of the patient, and in electrical contact with said stimulus-receiver means.

14. A method of claim 13, wherein said blocking is provided to at least one of left, right, or both vagus nerve(s), their branches or part thereof.

15. A system for providing electrical pulses for nerve blocking with or without selective electrical stimulation of the vagus nerve(s) and/or its branches or part thereof for treating, controlling or alleviating the symptoms for at least one of obesity, motility disorders, eating disorders, inducing weight loss, FGIDs, gastroparesis, gastro-esophageal reflex disease (GERD), pancreatitis, and ileus, comprising:
an external stimulator to generate and transmit blocking and/or stimulating electrical pulses;
an implanted stimulus-receiver means to inductively receive said electric pulses; and
a lead with at least two electrodes adapted to be in contact with said vagus nerve(s)or its branch(es) or part thereof in a patient and in electrical contact with said stimulus-receiver means.

16. A system of claim 15, wherein at least one electrode is providing said electric pulses for said nerve blocking.

17. A system of claim 15, wherein said nerve blocking is at least one of afferent blocking, efferent blocking, or organ block.

18. A system of claim 15, wherein said nerve blocking comprises at least one from a group consisting of: DC or anodal block, Wedenski block, and Collision block.

19. A system of claim 15, wherein said nerve blocking is provided to said at least one of the left and/or right vagus nerve(s) their branch(es) or part thereof at one or more sites.

20. The system of claim 15, wherein said external stimulator further comprises means for manually controlling and/or changing said electrical pulses.

21. The system of claim 15, wherein said implanted stimulus receiver means further comprises:
a) means for temporary power storage, and
b) said temporary power storage means is high value capacitors.

22. The system of claim 15, wherein said external stimulator comprises telemetry means for communication and data exchange over a wide area network to remotely interrogate and/or remotely program said external stimulator.

23. The system of claim 15, wherein a proximity sensing means is used to position said external coil with respect to said secondary coil.

24. The system of claim 15, wherein said implanted stimulus-receiver means comprises means for regulating said electric pulses that are provided to said nerve.

25. The system of claim 15, wherein said electrical pulses have predetermined parameter values of the variable components of said electric pulses comprising at least one of pulse amplitude, pulse width, pulse frequency, and on-off timing sequence such that, set(s) of said predetermined parameter values of the variable components of said electric pulses can be stored in memory of said external stimulator as program(s).
